(19) United States
(12) Patent Application Publication
(10) Pub. No.: US 2010/0069877 A1
(43) Pub. Date: Mar. 18, 2010

(54) APPARATUS AND METHOD TO DISPENSE HPC-BASED VISCOUS LIQUIDS INTO POROUS SUBSTRATES, E.G., CONTINUOUS WEB-BASED PROCESS

(76) Inventors: Gregory A. Smith, Sammamish, WA (US); Dale Kalamasz, Redmond, WA (US); Tyler D.J. Westcott, Seattle, WA (US); Robert P. Kinsey, Seattle, WA (US); Darrick Carter, Seattle, WA (US); Paul Sleath, Seattle, WA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 12/556,668

(22) Filed: Sep. 10, 2009

Related U.S. Application Data
(60) Provisional application No. 61/095,912, filed on Sep. 10, 2008.

(51) Int. Cl. A61M 5/00; A61K 31/167; A61P 23/02

(52) U.S. Cl. 604/500; 514/626

(57) ABSTRACT

Systems and methods are provided for dispensing compositions comprising sols, sol-forming compounds, or highly viscous compositions into porous substrates. In some embodiments, the porous substrates are elements of transdermal delivery devices. In some embodiments, the highly viscous compositions comprise alkylcellulose ethers, or derivatives thereof, in particular, hydroxypropyl cellulose. Such may be useful in continuous web-based manufacturing processes.

### BACKGROUND

1. Technical Field

This disclosure generally relates to the field of systems, devices and methods for transdermal delivery of drugs, including both active and passive delivery. This disclosure relates in particular to processes, for example, continuous web-based manufacturing processes, for making devices for use in drug delivery, e.g., transdermal drug delivery and more particularly to processes for filling porous substrates or drug reservoirs of such devices.

2. Description of the Related Art

Delivery of pharmaceutically active agents to or through a biological interface (e.g., skin, mucous membrane, and the like) can be a convenient, non-invasive, painless method for introducing drugs into dermal or sub-dermal tissues, or systemically throughout the body, of an organism. An active agent may include, e.g., a charged or uncharged substance, an ionized or non-ionized compound or drug, a therapeutic, a bioactive agent, and the like. Modes of delivery of pharmaceutically active agents to or through a biological interface may depend on the charge, ionic, or size characteristics of the agent. Pharmaceutically active agents may be delivered passively (e.g., by adsorption) or actively (e.g., by iontophoresis, electroporation, electrophoresis and/or electro-osmosis).

Passive delivery of a pharmaceutically active agent may be accomplished by applying a device to the biological interface. A passive delivery device may be in the form, e.g., of a simple active agent-containing patch or bandage and the like. Such devices contain the active agent or composition thereof and are designed to allow movement under passive conditions of the active agent from the interior or porous substrate of the device to and/or through the biological interface. Devices for passive delivery of pharmaceutical agents are used, for example, to locally deliver substances to control pain or promote healing or to systemically deliver agents to control the desire to smoke tobacco.

Iontophoretic delivery of pharmaceutically active agents employs an electromotive force and/or current to transfer the active agent to the biological interface by applying an electrical potential to an electrode proximate to an iontophoretic chamber containing a similarly charged pharmaceutically active agent and/or its vehicle or carrier.

Iontophoresis devices typically include an active electrode assembly and a counter electrode assembly, each coupled to opposite poles or terminals of a power source, for example, a chemical battery or an external power source. Each electrode assembly typically includes a respective electrode element to apply the electromotive force and/or current. Such electrodes often comprise a sacrificial element or compound, for example, silver or silver chloride. The active electrode assembly typically contains at least one active agent reservoir, which supplies the active agent for iontophoretic delivery. One or both electrode assemblies may also contain one or more electrolyte reservoirs. In an iontophoresis device, the active agent may be either cationic or anionic, and the power source may be configured to apply the appropriate voltage polarity based on the polarity of the active agent. An ion exchange membrane may be positioned in a device to serve as a polarity selective barrier between the portion of the device containing the active agent and the biological interface. The membrane, typically permeable only to one particular type of ion (e.g., a charged active agent), may prevent reverse flux from the skin or mucous membrane of ions having a charge opposite to those of the active agent. Iontophoresis may be advantageously used to enhance or control the delivery rate of the active agent compared to the rate that may be achieved using a passive delivery device.

In passive or active delivery devices, the portion of the device containing the pharmaceutically active agent may be a reservoir such as a cavity (see, e.g., U.S. Pat. No. 5,395,310). Alternatively, the active agent may be stored in a reservoir such as a gel matrix or a porous substrate, e.g., a fabric material, or some combination thereof. In particular, for example, when using a reservoir having a porous substrate, the reservoir may be filled, during manufacture, with some type of material, for example, a non-ionic hydrophilic polymer matrix, a sol, or a hydrogel.

Commercial acceptance of both passive and active delivery devices, including, in particular, iontophoresis devices, is dependent on a variety of factors, such as cost to manufacture, efficiency and/or uniformity and/or timeliness of active agent delivery, shelf life, stability during storage, biological capability, and/or disposal issues.

Accurate, consistent, efficient and cost-effective filling of the reservoirs, including active agent and electrolyte reservoirs, is necessary to satisfy certain of these needs. In certain filling methods, a reservoir support matrix is introduced into the reservoir and then allowed to dry. In a subsequent act, an aqueous active agent or electrolyte solution is then introduced and allowed to rehydrate the dried matrix, thus allowing the active agent or electrolyte solution to become absorbed into the matrix to form the active agent- or electrolyte-containing reservoir. Often in such a method, rehydration of the dried matrix is irregular and incomplete, thus preventing saturation of the matrix and yielding a non-uniform active agent or electrolyte concentration throughout the reservoir. Such non-uniformity of the reservoir may lead to inconsistent current flow and/or inconsistent delivery of active agent during use. Alternatively, the active agent and/or electrolyte and/or other reservoir components can be mixed into a solution or suspension containing polymers and/or other components, which may then be allowed to form a sol or a highly viscous solution containing the active agent and/or electrolyte and/or other components. The active agent-containing sol or highly viscous polymer solution may then be dispensed into the reservoir for absorption into the porous reservoir structure, thus forming the active agent- or electrolyte-containing reservoir. Although the concentration of active agent or electrolyte may be uniform throughout the sol or polymer matrix in this approach, the highly viscous consistency of the matrix, whether sol or polymer solution, can often lead to particular difficulty in uniformly dispersing the matrix throughout the porous substrate of the reservoir. Non-uniform dispensing and/or dispersal of the viscous matrix may lead to some of the same difficulties noted above when the device is put into use. Further, automated filling processes, such as continuous web-based processes, are often advantageous in manufacturing devices for administering active substances, but highly viscous materials are difficult to dispense in such processes. Accordingly, a system and method for efficiently and effectively dispensing viscous polymer matrix components containing active agent and/or electrolytes and/or any other desired components, including additives or excipients, into a porous substrate may be particularly advantageous.

The present disclosure addresses one or more of the shortcomings set forth above and/or provides further related advantages.

### BRIEF SUMMARY

This disclosure is directed to methods and systems for filling porous substrates or reservoirs, in particular, porous reservoir structures of various transdermal delivery devices, with highly viscous polymer compositions, sols, or sol-forming compounds, to produce reservoirs containing active agents and/or electrolytes and/or other compounds and/or excipients of interest for use in such delivery devices.

A method is provided for dispensing into a porous substrate a composition comprising a highly viscous liquid, a sol or a sol-forming material including at least one cellulose derivative via a conduit having an inlet, an outlet, a first portion spaced between the inlet and the outlet, and a second portion spaced between the first portion and the outlet. The method comprises providing at the outlet end of the conduit the porous substrate; adjusting a temperature of the first portion of the conduit to a first temperature of at least about 35° C., said temperature sufficient to transform a viscosity of the composition comprising the highly viscous liquid, the sol or the sol-forming material including at least one cellulose derivative from a high viscosity to a low viscosity; moving the composition comprising the highly viscous liquid, the sol or the sol-forming material including at least one cellulose derivative from the inlet end to the outlet end of the conduit; and dispensing the composition from the outlet end of the conduit into the porous substrate. In certain such embodiments, dispensing the composition from the outlet end of the conduit may include dispensing the composition having a viscosity that is between about 0 centipoise and about 500 centipoise. In other such embodiments, dispensing the composition from the outlet end of the conduit may include dispensing the composition having a viscosity that is between about 0 centipoise and about 200 centipoise. In further such embodiments, dispensing the composition from the outlet end of the conduit may include dispensing the composition having a viscosity that is between about 50 centipoise and about 150 centipoise. In yet other such embodiments, dispensing the composition from the outlet end of the conduit may include dispensing the composition having a viscosity that is between about 80 centipoise and about 120 centipoise.

The method may comprise adjusting a temperature of the first portion of the conduit to a first temperature of at least about 35° C. sufficient to transform a viscosity of the composition from a high viscosity of between about 2,500 centipoise and about 10,000 centipoise to a low viscosity. The method may comprise adjusting a temperature of the first portion of the conduit to a first temperature of at least about 35° C. sufficient to transform a viscosity of the composition from a high viscosity to a low viscosity of between about 0 centipoise and about 200 centipoise at some point in the first portion of the conduit. The method may comprise adjusting a temperature of the first portion of the conduit to a first temperature of at least about 35° C. sufficient to transform a viscosity of the composition from a high viscosity to a low viscosity of between about 50 centipoise and about 150 centipoise at some point in the first portion of the conduit. The method may comprise adjusting a temperature of the first portion of the conduit to a first temperature of at least about 35° C. sufficient to transform a viscosity of the composition from a high viscosity to a low viscosity of between about 80 centipoise and about 120 centipoise at some point in the first portion of the conduit.

The method may include adjusting the temperature of the first portion of the conduit to between about 45° C. and about 70° C. The method may include adjusting the temperature of the first portion of the conduit to between about 40° C. and about 60° C. Adjusting a temperature of the first portion of the conduit to a first temperature may include adjusting the temperature of the first portion to between about 40° C. and about 50° C. Adjusting a temperature of the first portion of the conduit to a first temperature may include adjusting the temperature of the first portion to between about 50° C. and about 60° C. Adjusting a temperature of the first portion of the conduit to a first temperature may include adjusting the temperature of the first portion to between about 45° C. and about 55° C. Adjusting a temperature of the first portion of the conduit to a first temperature may include adjusting the temperature of the first portion to between about 40° C. and about 43° C. Adjusting a temperature of the first portion of the conduit to a first temperature may include adjusting the temperature of the first portion to between about 43° C. and about 46° C. Adjusting a temperature of the first portion of the conduit to a first temperature may include adjusting the temperature of the first portion to between about 46° C. and about 49° C. Adjusting a temperature of the first portion of the conduit to a first temperature may include adjusting the temperature of the first portion to between about 49° C. and about 52° C. Adjusting a temperature of the first portion of the conduit to a first temperature may include adjusting the temperature of the first portion to between about 52° C. and about 55° C. Adjusting a temperature of the first portion of the conduit to a first temperature may include adjusting the temperature of the first portion to between about 55° C. and about 58° C. Adjusting a temperature of the first portion of the conduit to a first temperature may include adjusting the temperature of the first portion to between about 49° C. and about 53° C. Adjusting a temperature of the first portion of the conduit to a first temperature may include adjusting the temperature of the first portion to between about 39° C. and about 43° C.

A method is provided for dispensing into a porous substrate a composition comprising a highly viscous liquid, a sol or a sol-forming material including at least one cellulose derivative via a conduit having an inlet, an outlet, a first portion spaced between the inlet and the outlet, and a second portion spaced between the first portion and the outlet. The method comprises providing at the outlet end of the conduit the porous substrate; adjusting a temperature of the first portion of the conduit to a first temperature of at least about 35° C., said temperature sufficient to transform a viscosity of the composition comprising the highly viscous liquid, the sol or the sol-forming material including at least one cellulose derivative from a high viscosity of between about 2,500 centipoise and about 10,000 centipoise to a low viscosity; adjusting a temperature of the second portion of the conduit to a second temperature; moving the composition comprising the highly viscous liquid, the sol or the sol-forming material including at least one cellulose derivative from the inlet end to the outlet end of the conduit; and dispensing the composition from the outlet end of the conduit into the porous substrate.

Adjusting a temperature of the second portion of the conduit to a second temperature may include adjusting the temperature of the second portion sufficiently to maintain the viscosity of the composition at the low viscosity at some point in the second portion, e.g., at the outlet of the second portion. Adjusting the second temperature may include adjusting the temperature of the second portion such that the viscosity of the composition is between about 0 centipoise and about 200 centipoise at some point in the second portion. Adjusting the second temperature may include adjusting the temperature of the second portion such that the viscosity of the composition is between about 50 centipoise and about 150 centipoise at some point in the second portion. Adjusting the second temperature may include adjusting the temperature of the second portion such that the viscosity of the composition is between about 80 centipoise and about 120 centipoise at some point in the second portion. Adjusting a temperature of the second portion of the conduit to a second temperature may include adjusting the temperature of the second portion sufficiently to maintain the viscosity of the composition at the low viscosity at the outlet of the second portion.

Adjusting a temperature of the second portion of the conduit to a second temperature may include adjusting the temperature of the second portion to be greater than 35° C. Adjusting a temperature of the second portion of the conduit to a second temperature may include adjusting the temperature of the second portion to between about 35° C. and about 70° C. Adjusting a temperature of the second portion of the conduit to a second temperature may include adjusting the temperature of the second portion to between about 40° C. and about 50° C. Adjusting a temperature of the second portion of the conduit to a second temperature may include adjusting the temperature of the second portion to between about 50° C. and about 60° C. Adjusting a temperature of the second portion of the conduit to a second temperature may include adjusting the temperature of the second portion to between about 45° C. and about 55° C. Adjusting a temperature of the second portion of the conduit to a second temperature may include adjusting the temperature of the second portion to between about 40° C. and about 43° C. Adjusting a temperature of the second portion of the conduit to a second temperature may include adjusting the temperature of the second portion to between about 43° C. and about 46° C. Adjusting a temperature of the second portion of the conduit to a second temperature may include adjusting the temperature of the second portion to between about 46° C. and about 49° C. Adjusting a temperature of the second portion of the conduit to a second temperature may include adjusting the temperature of the second portion to between about 49° C. and about 52° C. Adjusting a temperature of the second portion of the conduit to a second temperature may include adjusting the temperature of the second portion to between about 52° C. and about 55° C. Adjusting a temperature of the second portion of the conduit to a second temperature may include adjusting the temperature of the second portion to between about 55° C. and about 58° C. Adjusting a temperature of the second portion of the conduit to a second temperature may include adjusting the temperature of the second portion to between about 49° C. and about 53° C. Adjusting a temperature of the second portion of the conduit to a second temperature may include adjusting the temperature of the second portion to between about 39° C. and about 43° C.

Adjusting a temperature of the second portion of the conduit to a second temperature may include adjusting the temperature of the second portion of the conduit to be about less than the temperature of the first portion of the conduit. Adjusting a temperature of the second portion of the conduit to a second temperature may include adjusting the temperature of the second portion of the conduit to be about the same as the temperature of the first portion of the conduit. Adjusting a temperature of the second portion of the conduit to a second temperature may include adjusting the temperature of the second portion of the conduit to be about greater than the temperature of the first portion of the conduit.

A method is provided for dispensing into a porous substrate a composition comprising a highly viscous liquid, a sol or a sol-forming material including at least one cellulose derivative via a conduit having an inlet, an outlet, a first portion spaced between the inlet and the outlet, and a second portion spaced between the first portion and the outlet. In one embodiment, the method comprises providing at the outlet end of the conduit the porous substrate; adjusting a temperature of the first portion of the conduit to a first temperature from about 52° C. to about 55° C.; adjusting a temperature of the second portion of the conduit to a second temperature from about 49° C. to about 52° C.; moving the composition comprising the highly viscous liquid, the sol or the sol-forming material including at least one cellulose derivative from the inlet end to the outlet end of the conduit; and dispensing the composition from the outlet end of the conduit into the porous substrate. In another embodiment, the method includes adjusting a temperature of the first portion of the conduit to a first temperature from about 49° C. to about 52° C. and adjusting a temperature of the second portion of the conduit to a second temperature from about 52° C. to about 55° C. In a further embodiment, the method includes adjusting a temperature of the first portion of the conduit and a temperature of the second portion of the conduit to about 49° C. to about 53° C. In a further embodiment, the method includes adjusting a temperature of the first portion of the conduit and a temperature of the second portion of the conduit to about 39° C. to about 43° C.

A method is provided for dispensing into a porous substrate a composition comprising a highly viscous liquid, a sol or a sol-forming material including at least one cellulose derivative via a conduit having an inlet, an outlet, a first portion spaced between the inlet and the outlet, and a second portion spaced between the first portion and the outlet. The method comprises providing the composition including the cellulose derivative in the form of at least one of an alkylcellulose ether or a modified alkylcellulose ether to the inlet of the conduit; providing at the outlet end of the conduit the porous substrate; adjusting a temperature of the first portion of the conduit to a first temperature of at least about 35° C., said temperature sufficient to transform a viscosity of the composition comprising the highly viscous liquid, the sol or the sol-forming material including at least one cellulose derivative from a high viscosity to a low viscosity; moving the composition comprising the highly viscous liquid, the sol or the sol-forming material including at least one cellulose derivative from the inlet end to the outlet end of the conduit; and dispensing the composition from the outlet end of the conduit into the porous substrate. The cellulose derivative may be in the form of at least one of a hydroxypropyl cellulose, a hydroxyethyl cellulose, a hydroxypropylmethyl cellulose, or a carboxymethyl cellulose. The cellulose derivative may be in the form of at least one of a derivative of hydroxypropyl cellulose, a derivative of hydroxyethyl cellulose, a derivative of hydroxypropylmethyl cellulose, or a derivative of carboxymethyl cellulose. The cellulose derivative may be in the form of a hydroxypropyl cellulose. The hydroxypropyl cellulose may be in a percent concentration (w/w×100) of from about 1% to about 2.5% to the inlet of the conduit. The hydroxypropyl cellulose may be in a percent concentration (w/w×100) of from about 1.5% to about 2% to the inlet of the conduit. The cellulose derivative may be in the form of a first cellulose derivative and a second cellulose derivative. The second cellulose derivative may be different from the first cellulose derivative. The first cellulose derivative may be hydroxypropyl cellulose. The second cellulose derivative may be different from hydroxypropyl cellulose. The cellulose derivative may be in the form of a mixture of hydroxypropyl cellulose and hydroxyethyl cellulose. In some embodiments, a ratio of percent concentration (w/w×100) of hydroxypropyl cellulose to percent concentration (w/w×100) of hydroxyethyl cellulose is from about 4:1 to about 2:1. In some embodiments, a ratio of percent concentration (w/w×100) of hydroxypropyl cellulose to percent concentration (w/w×100) of hydroxyethyl cellulose is from about 3.5:1 to about 2.5:1. In some embodiments, a ratio of percent concentration (w/w×100) of hydroxypropyl cellulose to percent concentration (w/×100) of hydroxyethyl cellulose is about 3:1.

The cellulose derivative may be in the form of a mixture of hydroxypropyl cellulose and hydroxyethyl cellulose, wherein the percent concentration of hydroxypropyl cellulose is about 1.5% and the percent concentration of hydroxyethyl cellulose is about 0.5%.

The composition including at least one cellulose derivative may further include at least one surfactant. The at least one surfactant may be a non-ionic surfactant, an ionic surfactant, an anionic surfactant, a cationic surfactant, or a zwitterionic surfactant. The non-ionic surfactant may be a poloxamer or a pluronic. The non-ionic surfactant may be selected from Poloxamer 188, Pluronic L 44, or Pluronic L 62. The non-ionic surfactant may be a polysorbate surfactant, e.g., TWEEN or SPAN.

A method is provided for dispensing into a porous substrate a composition comprising a highly viscous liquid, a sol or a sol-forming material including at least one cellulose derivative via a conduit having an inlet, an outlet, a first portion spaced between the inlet and the outlet, and a second portion spaced between the first portion and the outlet. The method comprises providing at the outlet end of the conduit the porous substrate; adjusting a temperature of the first portion of the conduit to a first temperature of at least about 35° C., said temperature sufficient to transform a viscosity of the composition comprising the highly viscous liquid, the sol or the sol-forming material including at least one cellulose derivative from a high viscosity to a low viscosity; moving the composition comprising the highly viscous liquid, the sol or the sol-forming material including at least one cellulose derivative from the inlet end to the outlet end of the conduit; and dispensing the composition from the outlet end of the conduit into the porous substrate; wherein the composition is in the form of an electrolyte composition.

A method is provided for dispensing into a porous substrate a composition comprising a highly viscous liquid, a sol or a sol-forming material including at least one cellulose derivative via a conduit having an inlet, an outlet, a first portion spaced between the inlet and the outlet, and a second portion spaced between the first portion and the outlet. The method comprises providing at the outlet end of the conduit the porous substrate; adjusting a temperature of the first portion of the conduit to a first temperature of at least about 35° C., said temperature sufficient to transform a viscosity of the composition comprising the highly viscous liquid, the sol or the sol-forming material including at least one cellulose derivative from a high viscosity to a low viscosity; moving the composition comprising the highly viscous liquid, the sol or the sol-forming material including at least one cellulose derivative from the inlet end to the outlet end of the conduit; and dispensing the composition from the outlet end of the conduit into the porous substrate; wherein the composition is in the form of a biologically active agent composition. The biologically active agent may, for example, be selected from the group consisting of—caine-type active agents. The active agent may be lidocaine. The active agent may be a lidocaine-containing composition further including epinephrine.

A method is provided for dispensing into a porous substrate a composition comprising a highly viscous liquid, a sol or a sol-forming material including at least one cellulose derivative via a conduit having an inlet, an outlet, a first portion spaced between the inlet and the outlet, and a second portion spaced between the first portion and the outlet. The method comprises providing the composition to the inlet of the conduit from a pressurized dispensing reservoir; providing at the outlet end of the conduit the porous substrate; adjusting a temperature of the first portion of the conduit to a first temperature of at least about 35° C., said temperature sufficient to transform a viscosity of the composition comprising the highly viscous liquid, the sol or the sol-forming material including at least one cellulose derivative from a high viscosity to a low viscosity; moving the composition comprising the highly viscous liquid, the sol or the sol-forming material including at least one cellulose derivative from the inlet end to the outlet end of the conduit; dispensing the composition from the outlet end of the conduit into the porous substrate; and regulating a flow of the composition from the outlet end of the conduit via a valve positioned at least proximate the second portion of the conduit. The composition may be dispensed into a holding portion of a device for delivery of an active agent to or through a biological interface. The composition may be dispensed into a holding portion of a device for transdermal delivery of an active agent to or through a biological interface. The composition may be dispensed into a holding portion of a device for iontophoretic delivery of an active agent to or through a biological interface and the composition, upon delivery, may be allowed to return to ambient temperature. The composition may be dispensed into a porous substrate-containing matrix within the holding portion of the device.

A method is provided for dispensing into a porous substrate a composition comprising a highly viscous liquid, a sol or a sol-forming material including at least one cellulose derivative via a conduit having an inlet, an outlet, a first portion spaced between the inlet and the outlet, and a second portion spaced between the first portion and the outlet. The method comprises providing the composition to the inlet of the conduit from a metering pump, e.g., a positive displacement pump; providing at the outlet end of the conduit the porous substrate; adjusting a temperature of the first portion of the conduit to a first temperature of at least about 35° C., said temperature sufficient to transform a viscosity of the composition comprising the highly viscous liquid, the sol or the sol-forming material including at least one cellulose derivative from a high viscosity to a low viscosity; moving the composition comprising the highly viscous liquid, the sol or the sol-forming material including at least one cellulose derivative from the inlet end to the outlet end of the conduit; dispensing the composition from the outlet end of the conduit into the porous substrate; and regulating a flow of the composition from the outlet end of the conduit by adjusting the metering pump to regulate flow of the composition to the inlet and thereby through the conduit to the outlet. The composition may be dispensed into a holding portion of a device for delivery of an active agent to or through a biological interface. The composition may be dispensed into a holding portion of a device for transdermal delivery of an active agent to or through a biological interface. The composition may be dispensed into a holding portion of a device for iontophoretic delivery of an active agent to or through a biological interface and the composition, upon delivery, may be allowed to return to ambient temperature. The composition may be dispensed into a porous substrate-containing matrix within the holding portion of the device.

A system is provided for dispensing a highly viscous liquid, a sol or a sol-forming composition comprising at least one cellulose derivative. The system comprises: a conduit for transporting the composition, the conduit comprising an inlet, an outlet, a first portion positioned between the inlet and the outlet and a second portion positioned between the first portion and the outlet; a first heater positioned to heat the composition in at least part of the first portion of the conduit to a first temperature; and a valve mechanism operable to control a rate of dispensing the composition from the conduit. The system may further include a second heater positioned to heat the composition in at least part of the second portion of the conduit to a second temperature. The first heater may include at least one of a heat exchanger or a heater element. The second heater may include at least one of a heat exchanger or a heater element. The second temperature may be less than or equal to the first temperature. The second temperature may be greater than or equal to the first temperature. At least a portion of the second heater may be embedded in at least a portion of the valve mechanism.

A system is provided for dispensing a highly viscous liquid, a sol or a sol-forming composition comprising at least one cellulose derivative. The system comprises: a conduit for transporting the composition, the conduit comprising an inlet, an outlet, a first portion positioned between the inlet and the outlet and a second portion positioned between the first portion and the outlet; a first heater positioned to heat the composition in at least part of the first portion of the conduit to a first temperature; a mixer at least proximate the first portion; and a valve mechanism operable to control a rate of dispensing the composition from the conduit. In certain embodiments, the mixer may be a static mixer. In other embodiments, the mixer may be a dynamic mixer.

A reservoir may be provided to store the composition to be dispensed, the reservoir fluidly communicably coupled to the inlet of the conduit via a fluid-tight connection. The reservoir may be a pressurized reservoir. The reservoir may store the composition that includes the cellulose derivative in the form of a hydroxypropyl cellulose. The reservoir may store a composition that includes the cellulose derivative in the form of a mixture of hydroxypropyl cellulose and hydroxyethyl cellulose.

The reservoir may store a composition that further comprises at least one biologically active agent. The reservoir may store a composition that further comprises at least one biologically active agent selected from the—caine-class active agents.

In certain embodiments of the systems disclosed herein, the at least one cellulose derivative is hydroxypropyl cellulose. In other embodiments of the systems disclosed here, the at least one cellulose derivative is a derivative of hydroxypropyl cellulose.

A system is provided for dispensing a highly viscous liquid, a sol or a sol-forming composition comprising at least one cellulose derivative. The system comprises: a conduit for transporting the composition, the conduit comprising an inlet, an outlet, a first portion positioned between the inlet and the outlet and a second portion positioned between the first portion and the outlet; a metering pump to supply the composition to the inlet of the conduit; and a first heater positioned to heat the composition in at least part of the first portion of the conduit to a first temperature. The system may further include a second heater positioned to heat the composition in at least part of the second portion of the conduit to a second temperature. The first heater may include at least one of a heat exchanger or a heater element. The second heater may include at least one of a heat exchanger or a heater element. The second temperature may be less than or equal to the first temperature. The second temperature may be greater than or equal to the first temperature.

A system is provided for dispensing a highly viscous liquid, a sol or a sol-forming composition comprising at least one cellulose derivative. The system comprises: a capillary tubing for transporting the composition, the capillary tubing comprising an inlet, an outlet, a first portion positioned between the inlet and the outlet and a second portion positioned between the first portion and the outlet; and a first heater positioned to heat the composition in at least part of the first portion of the capillary tubing to a first temperature. The system may further include a second heater positioned to heat the composition in at least part of the second portion of the capillary tubing to a second temperature. The first heater may include at least one of a heat exchanger or a heater element. The second heater may include at least one of a heat exchanger or a heater element. The second temperature may be less than or equal to the first temperature. The second temperature may be greater than or equal to the first temperature. In certain such embodiments, the composition may be supplied to the inlet of the capillary tubing via a pressurized reservoir. In other such embodiments, the composition may be supplied to the inlet of the capillary tubing via a metering pump.

A composition is provided comprising: a first cellulose derivative; a second cellulose derivative; and a biologically active agent; wherein the first cellulose derivative is hydroxypropyl cellulose; and wherein the second cellulose derivative differs from the first cellulose derivative. The second cellulose derivative may be selected from hydroxyethyl cellulose, hydroxypropylmethyl cellulose, or carboxymethyl cellulose. The second cellulose derivative may be a derivative of hydroxyethyl cellulose, a derivative of hydroxypropylmethyl cellulose, or a derivative of carboxymethyl cellulose. The second cellulose derivative may be hydroxyethyl cellulose. The biologically active agent may be selected from the group consisting of the—caine-type active agents. The biologically active agent may be lidocaine or a mixture of lidocaine and epinephrine.

A composition is provided comprising: a first cellulose derivative; a second cellulose derivative; and a biologically active agent; wherein the first cellulose derivative is a derivative of hydroxypropyl cellulose; and wherein the second cellulose derivative differs from the first cellulose derivative. The second cellulose derivative may be selected from hydroxyethyl cellulose, hydroxypropylmethyl cellulose, or carboxymethyl cellulose. In certain embodiments, the second cellulose derivative may be a derivative of hydroxyethyl cellulose, a derivative of hydroxypropylmethyl cellulose, or a derivative of carboxymethyl cellulose. The second cellulose derivative may be hydroxyethyl cellulose. The biologically active agent may be selected from the group consisting of the—caine-type active agents. The biologically active agent may be lidocaine or a mixture of lidocaine and epinephrine.

The compositions may be used in any of the methods and systems disclosed elsewhere herein.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1 is an isometric view of an active side of a passive transdermal delivery device according to one illustrated embodiment.

FIG. 2A is a top plan view of the active side of the passive transdermal delivery device of FIG. 1 according to one illustrated embodiment.

FIG. 2B is an exploded elevational view of the active side of the passive transdermal delivery device of FIG. 1 according to one illustrated embodiment.

FIG. 3 is a top plan view of an iontophoresis transdermal delivery device comprising an active electrode assembly and a counter electrode assembly according to one illustrated embodiment.

FIG. 4 is a top plan view of an iontophoresis transdermal delivery device comprising an active electrode assembly and a counter electrode assembly according to another illustrated embodiment.

FIG. 5A is a schematic diagram of an iontophoresis device according to one illustrated embodiment.

FIG. 5B is a schematic diagram of an iontophoresis device according to another illustrated embodiment.

FIG. 6A is a schematic view of a system for dispensing composition into a continuous web-based porous substrate according to one illustrated embodiment.

FIG. 6B is an isometric view of a portion of FIG. 6A showing a heater and a mixer as separate elements according to one illustrated embodiment.

FIG. 6C is an isometric view of a portion of FIG. 6A showing a heater and a mixer combined as a single element according to one illustrated embodiment.

FIG. 6D is a schematic view of an alternative form of the system of Figure A showing a metering pump and a second mixer according to one illustrated embodiment.

FIG. 6E is an expanded schematic view of a portion of FIG. 6A showing an apparatus to supply and fill a continuous web-based porous substrate and to prepare passive transdermal delivery devices therefrom according to one illustrated embodiment.

FIG. 6F is an expanded schematic view of a portion of FIG. 6A showing an apparatus to supply and fill a continuous web-based porous substrate and to prepare active transdermal delivery devices therefrom according to one illustrated embodiment.

FIG. 6G is an expanded schematic view of a portion of FIG. 6F showing an apparatus to supply and fill two continuous web-based porous substrates suitable to prepare active transdermal delivery device electrode assemblies having two filled reservoirs according to one illustrated embodiment.

FIG. 7 is an isometric view of a fill system for dispensing matrix into individual reservoirs of transdermal delivery devices according to one illustrated embodiment.

FIG. 8 is a flow diagram of a method for dispensing a cellulose derivative-containing composition through a conduit into a porous substrate, wherein the composition is heated before dispensing, according to one illustrated embodiment.

FIG. 9 is a flow diagram of a method for dispensing a cellulose derivative-containing composition through a conduit into a porous substrate, wherein the composition is heated before dispensing, according to one illustrated embodiment.

FIG. 10 is a flow diagram of a method for dispensing a cellulose derivative-containing composition into a porous substrate, wherein the composition is provided to an inlet of the conduit, according to one illustrated embodiment.

FIG. 11 is a flow diagram of a method for dispensing a cellulose derivative-containing composition into a porous substrate, wherein the rate at which the composition is dispensed is regulated via a valve, according to one illustrated embodiment.

FIG. 12 is a flow diagram of a method for dispensing a cellulose derivative-containing composition into a porous substrate, wherein the dispensed composition is allowed to return to ambient temperature, according to one illustrated embodiment.

FIG. 13A is a graph showing a plot of viscosity versus temperature of an iontophoresis counter electrode solution comprising an electrolyte in a mixture of hydroxypropyl cellulose and hydroxyethyl cellulose in one illustrated embodiment.

FIG. 13B is a graph showing a plot of viscosity versus temperature of an iontophoresis active electrode solution comprising a lidocaine active agent in a mixture of hydroxypropyl cellulose and hydroxyethyl cellulose in one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with delivery devices including, but not limited to, voltage and/or current regulators, or protective coverings and/or liners to protect delivery devices during shipping and storage, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment,” or “an embodiment,” or “another embodiment,” or “some embodiments,” or “certain embodiments” means that a particular referent feature, structure, or characteristic described in connection with the embodiment is included in at least one embodiment. Thus, the appearance of the phrases “in one embodiment,” or “in an embodiment,” or “in another embodiment,” or “in some embodiments,” or “in certain embodiments” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an iontophoretic delivery device for delivering an active agent includes a single species of active agent, but may also include multiple species of active agents in the single device. References to an active agent is also understood to include analogs and/or derivatives thereof. It should also be noted that the term “or” is generally employed as including “and/or” unless the content clearly dictates otherwise.

As used herein, the term “transdermal delivery” of an active agent refers to passive diffusion or active transport of the active agent through a biological interface, such as a skin or mucous membrane, wherein active transport results from application of an electromotive force and/or current. In this sense, a “transdermal delivery device” is a device that delivers an active agent through a biological interface. A “passive transdermal delivery device” passively delivers an active agent; an “active transdermal delivery device” actively delivers an active agent.

As used herein, the term “reservoir” means any form or mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more porous membranes, substrates or structures; ion-exchange membranes, substrates or structures; semi-permeable membranes, substrates or structures; and/or sols, if such are capable of at least temporarily retaining an element or compound. Porous substrates or structures may include various types of fabrics and/or other fibrous materials. Typically, a reservoir serves to retain a biologically active agent prior to the discharge of such an agent by passive diffusion or by electromotive force and/or current to, into or through a biological interface. A reservoir may alternatively or additionally retain an electrolyte.

As used herein, the term “active agent” refers to a compound, molecule, or treatment that elicits a biological response from any host, animal, vertebrate, or invertebrate, including for example mammals, amphibians, reptiles, birds, fish, and humans. Examples of active agents include therapeutic agents, pharmaceutical agents, pharmaceuticals (e.g., a drug, a therapeutic compound, pharmaceutical salts, and the like), non-pharmaceuticals (e.g., cosmetic substances, and the like), a vaccine, an immunological agent, a local or general anesthetic or painkiller, an antigen or a protein or peptide, such as insulin, a chemotherapy agent, and/or an anti-tumor agent.

In some embodiments, the term “active agent” further refers not only to the active agent, but also to its pharmacologically active salts, pharmaceutically acceptable salts, prodrugs, metabolites, analogs, derivatives, and the like. In some further embodiments, the active agent includes at least one ionic, cationic, anionic, ionizable and/or neutral therapeutic drug and/or pharmaceutically acceptable salts thereof. In yet other embodiments, the active agent may include one or more “cationic active agents” that are positively charged and/or are capable of forming positive charges in aqueous media. For example, many biologically active agents have functional groups that are readily convertible to a positive ion or can dissociate into a positively charged ion and a counter ion in an aqueous medium. For instance, an active agent having an amino group can typically take the form of an ammonium salt in solid state and dissociate into a free ammonium ion (NH₄⁺) in an aqueous medium of appropriate pH. In further embodiments, the active agent may include one or more “anionic active agents” that are negatively charged and/or are capable of forming negative charges in aqueous media. For example, biologically active agents may have functional groups that are readily convertible to a negative ion or can dissociate into a negatively charged ion and a counter ion in an aqueous medium. In yet other embodiments, the active agents may be polarized or polarizable, that is, exhibiting a polarity at one portion relative to another portion.

The term “active agent” may also refer to electrically neutral agents, molecules, or compounds capable of being delivered by diffusion from a passive transdermal delivery device or by being carried by the flow of, for example, a solvent during iontophoresis, e.g., by electro-osmosis. Selection of suitable active agents is therefore within the knowledge of one skilled in the relevant art.

In some embodiments, one or more active agents can be selected from analgesics, anesthetics, vaccines, antibiotics, adjuvants, immunological adjuvants, immunogens, tolerogens, allegens, toll-like receptor agonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include lidocaine, articaine, and others of the—caine class; morphine, hydromorphone, fentanyl, oxycodone, hydrocodone, buprenorphine, methadone, and similar opioid agonists; sumatriptan succinate, zolmitriptan, naratriptan HCI, rizatriptan benzoate, almotriptan malate, frovatriptan succinate and other 5-hydroxytryptamine1 receptor subtype agonists; resiquimod, imiquimod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of anesthetic active agents or pain killers include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, the term “subject” generally refers to any host, animal, vertebrate, or invertebrate, and includes fish, mammals, amphibians, reptiles, birds, and particularly humans.

As used herein and in the claims, the term “biological interface” refers to both skin and mucous membrane (such as nasal membrane). Unless specified otherwise, all descriptions pertaining to delivery to or through the skin also apply to mucosal membranes.

As used herein, the term “effective amount” or “therapeutically effective amount” includes an amount effective at dosages and for periods of time necessary, to achieve the desired result. The effective amount of a composition containing a pharmaceutical agent may vary according to factors such as the disease state, age, gender, and weight of the subject.

As used herein, the term “analgesic” refers to an agent that lessens, alleviates, reduces, relieves, or extinguishes a neural sensation in an area of a subject's body. In some embodiments, the neural sensation relates to pain, in other aspects the neural sensation relates to discomfort, itching, burning, irritation, tingling, “crawling,” tension, temperature fluctuations (such as fever), inflammation, aching, or other neural sensations.

As used herein, the term “anesthetic” refers to an agent that produces a reversible loss of sensation in an area of a subject's body. In some embodiments, the anesthetic is considered to be a “local anesthetic” in that it produces a loss of sensation only in one particular area of a subject's body.

As one skilled in the relevant art would recognize, some agents may act as both an analgesic and an anesthetic, depending on the circumstances and other variables including but not limited to dosage, method of delivery, medical condition or treatment, and an individual subject's genetic makeup. Additionally, agents that are typically used for other purposes may possess local anesthetic or membrane stabilizing properties under certain circumstances or under particular conditions.

As used herein, the term “immunogen” refers to any agent that elicits an immune response. Examples of an immunogen include, but are not limited to natural or synthetic (including modified) peptides, proteins, lipids, oligonucleotides (RNA, DNA, etc.), chemicals, or other agents.

As used herein, the term “allergen” refers to any agent that elicits an allergic response. Some examples of allergens include but are not limited to chemicals and plants, drugs (such as antibiotics, serums), foods (such as milk, wheat, eggs, etc), bacteria, viruses, other parasites, inhalants (dust, pollen, perfume, smoke), and/or physical agents (heat, light, friction, radiation). As used herein, an allergen may be an immunogen.

As used herein, the term “adjuvant” and any derivations thereof, refers to an agent that modifies the effect of another agent while having few, if any, direct effect when given by itself. For example, an adjuvant may increase the potency or efficacy of a pharmaceutical, or an adjuvant may alter or affect an immune response.

As used herein, the terms “vehicle,” “carrier,” “pharmaceutical vehicle,” “pharmaceutical carrier,” “pharmaceutically acceptable vehicle,” or “pharmaceutically acceptable carrier” may be used interchangeably, and refer to pharmaceutically acceptable solid or liquid, diluting or encapsulating, filling or carrying agents, which are usually employed in pharmaceutical industry for making pharmaceutical compositions. Examples of vehicles include any liquid, sol, gel, salve, cream, solvent, diluent, fluid ointment base, vesicle, liposomes, niosomes, ethasomes, transfersomes, virosomes, cyclic oligosaccharides, non-ionic surfactant vesicles, phospholipid surfactant vesicles, micelle, and the like, that is suitable for use in contacting a subject.

In some embodiments, the pharmaceutical vehicle may refer to a composition that includes and/or delivers a pharmacologically active agent, but is generally considered to be otherwise pharmacologically inactive. In some other embodiments, the pharmaceutical vehicle may have some therapeutic effect when applied to a site such as a mucous membrane or skin, by providing, for example, protection to the site of application from conditions such as injury, further injury, or exposure to elements. Accordingly, in some embodiments, the pharmaceutical vehicle may be used for protection without a pharmacological agent in the formulation.

As used herein, the term “front surface” or “front side” generally refers, unless otherwise specified, to a side of an element nearest to, or designed to be nearest to, a biological interface of a living body. The front surface or front side is also referred to as the proximal surface or the proximal side, or as being proximal to the biological surface. As used herein, the term “back surface” or “back side” generally refers, unless otherwise specified, to a side of an element furthest from, or designed to be furthest from, a biological interface of a living body. The back surface or back side is also referred to as the distal surface or the distal side, or as being distal to the biological surface. Thus, for example, a proximal and a distal surface of a porous substrate are, respectfully, the surface nearest to the biological interface and the surface farthest from the biological interface.

As used herein and in the claims, the term “surfactant” refers to a surface active agent or wetting agent. A surfactant acts to lower surface tension of a liquid, in particular water, thus allowing the liquid to spread more easily. For example, a surfactant-containing aqueous composition may more efficiently enter into and spread throughout a porous membrane, substrate or structure. Surfactant molecules are typically amphipathic organic molecules, that is, having both hydrophilic and hydrophobic groups. Surfactants reduce the surface tension of an aqueous solution or suspension by arranging at the air-water interface. Surfactants may also arrange at an oil-water interface.

Surfactants may be either non-ionic or ionic. Non-ionic surfactants are particularly suitable for use in iontophoretic methods and devices. Non-limiting examples of non-ionic surfactants include polyoxypropylene-polyoxyethylene block copolymers, also known as poloxamers, poloxamines, or pluronics, e.g., PLURONIC L 44, PLURONIC L 62 and POLOXAMER 188; alkylpolyethylene oxides; alkylphenolpolyethylene oxides, e.g., TRITON X-100; and polysorbates, e.g., TWEEN and SPAN.

Ionic surfactants may be anionic, cationic or zwitterionic (amphoteric). Non-limiting examples of ionic surfactants include sodium dodecyl sulfate, perfluorooctanoate, perfluorooctanesulfonate, lauryl dimethylamine oxide, alkyl benzene sulfonate, fatty acid salts, alkyltrimethylammonium salts, e.g., cetyltrimethylammonium bromide, cetylpyridinium chloride, benzalkonium chloride, benzethonium chloride, methylbenzethonium chloride, dodecyl betaine, and cocamidopropyl betaine.

Further non-limiting examples of surfactants include emulsifying agents, amphoteric surfactants, non-ionic surfactants, ionic surfactants, acetone-insoluble phosphatides, phospholipids, amphiphiles, biocompatible surfactants, ether lipids, fluoro-lipids, polyhydroxyl lipids, polymerized liposomes, lecithin, hydrogenated lecithin, naturally occurring lecithin, egg lecithin, hydrogenated egg lecithin, soy lecithin, hydrogenated soy lecithin, vegetable lecithin, sorbitan esters, sorbitant monoesters, sorbitan monolaurate, sorbitan monooleate, sorbitan monopalmitate, sorbitan monostearate, sorbitan monostearate-palmitate, sorbitan sexquioleate, sorbitan tristearate, sorbitan trioleate, diacylglycerols, gangliosides, glycerophospholipids, lysophospholipids, mixed-chain phospholipids, pegylated phospholipids, phosphatidic acids, phosphatidylcholines, phosphatidylethanolamines, phosphatidylinositols, phosphocholines, phosphoethanolamines, phosphoglycerols, phosphoserines, phytosphingosines, sphingosines, and the like, or combinations thereof.

As used herein and in the claims, the term “cellulosic polymer” refers to a polymer having as its primary component a cellulose molecule. A cellulosic polymer includes, for example, a modified cellulose or a cellulose analog or derivative, and the like. Cellulosic polymers may include cellulose ethers or cellulose esters. Cellulose ethers include alkylcellulose ethers or modified alkylcellulose ethers, such as ethyl cellulose, methyl cellulose, hydroxypropyl cellulose, hydroxyethyl cellulose, hydroxyethylmethyl cellulose, hydroxypropylmethyl cellulose, and hydroxymethyl cellulose. Cellulose esters include, for example, cellulose acetate or cellulose triacetate. Cellulosic polymers may also include ionic cellulose ethers, such as carboxymethyl cellulose, or ionic cellulose esters, such as nitrocellulose.

As used herein and in the claims, the term “viscosity” refers to the degree to which a fluid resists flow under the influence of an applied force, termed shear stress, that is, stress applied tangential or parallel to the surface of a fluid. In other words, viscosity is a measure of the internal friction of a fluid caused by molecular attraction within the fluid. The rate at which two parallel planes of fluid move relative to one another is termed the shear rate. The fundamental unit of viscosity is the poise (100 centipoise), which is calculated by dividing the shear stress, i.e., the force required, by the resulting shear rate, i.e., the velocity produced. Viscosity is measured using a viscometer. A highly viscous fluid requires greater force, or shear stress, to produce a given flow, or shear rate, than that required to move less viscous fluids. Because viscosity relates to molecular attractions within a fluid, the composition of the fluid, the nature of the flow, and the characteristics and operating parameters of the viscometer may affect the viscosity measured. For example, under extreme shear conditions, measured viscosity may vary considerably from that measured under conditions generally recommended by a viscometer's manufacturer.

The headings provide herein are for convenience only and do not interpret the scope or meaning of the embodiments.

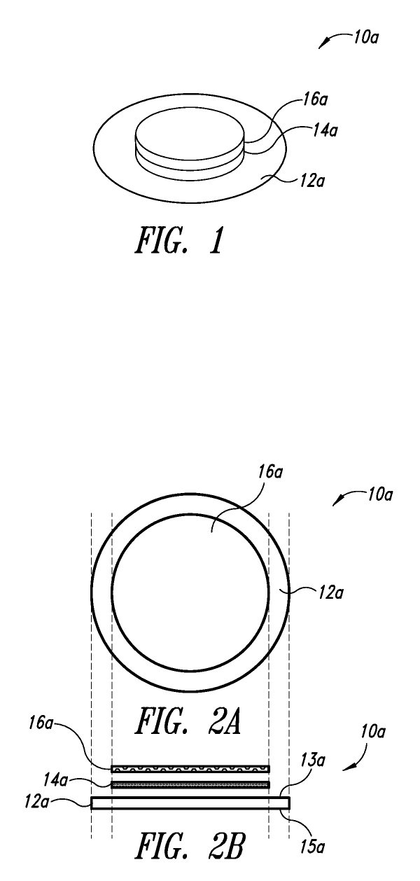

FIGS. 1, 2A, and 2B show an exemplary embodiment of a passive transdermal delivery device 10a. In some embodiments, the delivery device 10a is configured to transdermally deliver one or more therapeutic active agents to a biological interface of a subject via passive diffusion. The delivery device 10a in this illustrated embodiment includes a backing substrate 12a having opposed sides 13a and 15a. An optional base layer 14a is disposed and/or formed on the side 13a of the backing substrate 12a. An active agent layer 16a is disposed and/or formed on a back side of the base layer 14a. The backing substrate 12a, the optional base layer 14a, and the active agent layer 16a may be formed from pliable materials such that the delivery device 10a can conform to the contours of the subject. The active agent layer 16a may comprise a porous substrate containing the active agent and may further comprise a highly viscous liquid or sol, such as a high viscosity HPC-containing sol.

FIG. 1 shows an isometric view of the passive transdermal delivery device 10a. When the delivery device 10a is placed on the subject (not shown), the active agent layer 16a is proximal to the subject, and the backing substrate 12a is distal to the subject. The backing substrate 12a may include an adhesive such that the delivery device 10a may be applied to the subject and be adhered thereon. In some embodiments, the backing 12a encases the delivery device 10a. Non-limiting examples of backing substrates include 3M™ CoTran™ Backings, 3M™ CoTran™ Nonwoven Backings, and 3M™ Scotchpak™ Backings.

The optional base layer 14a may be constructed out of any suitable material including, for example, polymers, thermoplastic polymer resins (e.g., poly(ethylene terephthalate)), and the like. In some embodiments, the optional base layer 14a and the active agent layer 16a may cover a substantial portion of the backing substrate 12a. For example, in some embodiments, the backing substrate 12a, the optional base layer 14a, and the active agent layer 16a may be disk shaped and the backing substrate 12a may have a diameter of about 15 millimeter (mm) and the optional base layer 14a and the active agent layer 16a may have respective diameters of about 12 mm. In some embodiments, the sizes of the backing substrate 12a, the base layer 14a, and the active agent layer 16a may be larger or smaller, and in some embodiments, the relative size differences between the backing substrate 12a, the base layer 14a, and the active agent layer 16a may be different from that shown in FIGS. 1, 2A, and 2B. In some embodiments, the size of the active agent layer 16a may depend upon, among other things, the active agent or active agents being delivered by the delivery device 10a and/or the rate at which the active agent or active agents are to be delivered by the delivery device 10a. Typically, the backing substrate 12a and the base layer 14a are sized to the active agent layer 16a such that the sizes of the backing substrate 12a and the base layer 14a are at least the size of the active agent layer 16a.

Passive delivery devices are not limited to devices similar to those exemplified above but may also include any device having a porous substrate and designed to be applied directly or indirectly to a biological interface, such as pads, bandages (with or without adhesive), and the like. Any such devices may be advantageously filled with compositions, such as drug-containing compositions, according to methods described herein.

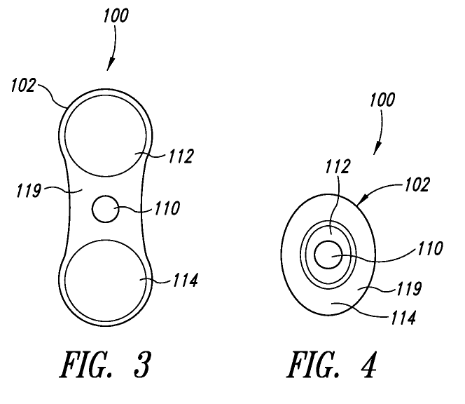

FIGS. 3 and 4 show exemplary active transdermal delivery systems 100 for delivery of one or more active agents to a subject (not shown) via iontophoresis, electroporation, electrophoresis and/or electro-osmosis. For convenience, the active transdermal delivery systems 100 will be generally discussed as iontophoresis systems, although in reality the precise mode of active delivery may not be important or even discernable. The iontophoresis systems 100 in the illustrated embodiments include an iontophoresis device 102 including active and counter electrode assemblies 112, 114, respectively, and a portable power supply system 110. The overall shape of the iontophoresis device 102 may take a variety of geometric forms including, for example, those shown in FIGS. 3 and 4.

In some embodiments, the active electrode assembly 112 takes the form of a positive electrode assembly, and the counter electrode assembly 114 takes the form of a negative electrode assembly. Alternatively, the active electrode assembly 112 may take the form of a negative electrode assembly, and the counter electrode assembly 114 may take the form of a positive electrode assembly. The active and counter electrode assemblies 112, 114 are electrically coupleable to the portable power supply system 110 to supply an active agent contained in the active electrode assembly 112, via iontophoresis, to a biological interface.

The transdermal delivery device 102 may optionally include a backing 119. In some embodiments, the backing 119 encases the iontophoresis device 102. In some other embodiments, the backing 119 physically couples the transdermal delivery device 102 to a biological interface of a subject. In some embodiments, the transdermal delivery system 102 is configured to provide transdermal delivery of one or more therapeutic active agents to a biological interface of a subject.

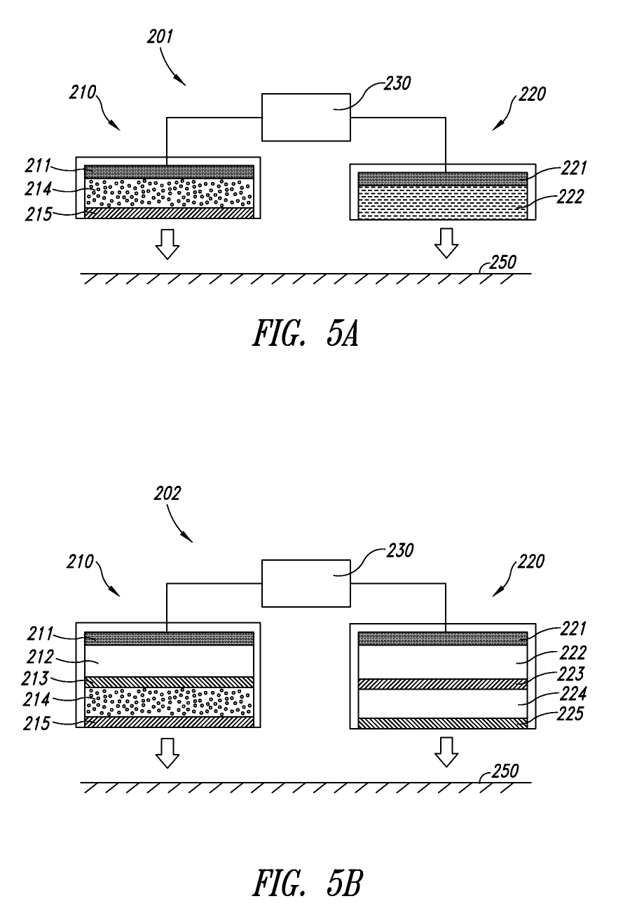

FIGS. 5A and 5B show schematic diagrams of exemplary iontophoresis transdermal delivery devices displaying exemplary interior elements of the devices. FIG. 5A in a particular illustrated embodiment shows an iontophoresis device 201, which includes (1) an active electrode assembly 210 having an active electrode 211 with a first polarity, an active agent reservoir 214 for holding an active agent having the first polarity, and optionally an ion-exchange membrane 215, having a charge of the first polarity; (2) a counter electrode assembly 220 having a counter electrode 221 with a second polarity opposite to that of the first polarity and an electrolyte reservoir 222 for holding an electrolyte solution in contact with the counter electrode 221; and (3) a power source 230 having terminals connected to active electrode 211 and counter electrode 221. In at least one embodiment, during operation of the device 200, the poles of the power source 230, having a first polarity and a second polarity, are connected respectively to the active electrode assembly 210 and the counter electrode assembly 220; the device 200 is brought into contact with a biological interface 250 of a subject; and, upon activation of the power source 230, active agent of a first polarity migrates from the active agent reservoir 214 to and into the biological interface 250. In certain such embodiments, the device 201 may include the optional ion-exchange membrane 215 in contact with the biological interface 250, which may serve to substantially block the migration of biological counter ions from the biological interface 250 into the active electrode assembly 210, thereby improving the efficiency of delivery of the active agent.

FIG. 5B shows an iontophoresis device 202 according to another illustrated embodiment, which includes elements in addition to those shown in device 201. Device 202 further includes, in the active electrode assembly 210, an electrolyte reservoir 212 for holding an electrolyte solution in contact with the active electrode 211 and the active agent reservoir 214. In certain embodiments, an optional second ion-exchange membrane 213 having the second polarity may be positioned between the front side of the electrolyte reservoir 212 and the back side of the active agent reservoir 214. In the particular illustrated embodiment in FIG. 5B, device 202 further includes, as elements of the counter electrode assembly 220, a further electrolyte reservoir 224 and two optional ion-exchange membranes 223, 225, with membrane 223 positioned between electrolyte reservoirs 222 and 224 and with membrane 225 positioned between electrolyte reservoir 224 and biological interface 250.

Reservoirs in devices, such as the transdermal delivery devices described above, may include, for example, porous substrates or membranes into which sol-like materials, including highly viscous polymer solutions, containing active agents and/or electrolytes, and the like, as well as other components or excipients, are dispersed. Such reservoirs may be particularly useful to provide consistent, uniform migration of active agents and/or electrolytes under passive or active conditions of use. The sol-like materials may comprise semi-solid suspensions or solutions of organic macromolecules. The solutions may be highly viscous. Examples of such organic macromolecules include cellulosic polymers (e.g., hydroxypropyl cellulose, hydroxyethyl cellulose, hydroxypropyl methyl cellulose, methyl cellulose, and the like), gelling agents (carboxypolyalkylenes, and the like), hydrophilic polymers (e.g., polyethylene oxides, polyoxyethylene-polyoxypropylene copolymers, polyvinylalcohols, and the like), gelatin, xanthan gums, sodium alginate, and the like, or combinations thereof.

Cellulosic polymers, for example hydroxypropyl cellulose, may be particularly useful materials for dispersal throughout porous substrates or membranes, to provide a medium for stably holding the active agent and/or electrolyte, as well as other compounds or excipients. As discussed elsewhere herein, however, there are challenges in providing a porous reservoir structure within which stable sol or cellulosic polymer compositions containing uniform concentrations of active agent and/or electrolyte are dispersed throughout the porous substrate.

Two approaches that may be taken to attempt to prepare porous substrates throughout which are dispersed cellulosic polymer compositions containing uniform concentrations of active agents and/or electrolytes are as follows.

In one approach to filling porous substrates suitable for preparation of reservoirs of devices described herein, and related devices, the active agent and/or electrolyte, as well as other compound(s) and/or expedient(s), may be mixed into solution with the cellulosic polymer component(s) during preparation of the polymer solution. The cellulosic polymer solution, which becomes highly viscous upon dissolution of the polymer component(s), thus may contain a well-distributed uniform concentration of the active agent and/or electrolyte, as well as other compounds and/or expedients. However, upon attempting to dispense such highly viscous polymer solutions into a porous substrate, the viscous solution can be dispensed only with difficulty and further, when dispensed, is unable to flow readily into and throughout the porous substrate, thus yielding a device reservoir that contains a non-uniformly distributed solution of cellulosic polymer containing active agent and/or electrolyte. Thus, when used for transdermal delivery of an active agent, a device having reservoirs comprising porous substrate filled in this manner is likely to display irregularities in the flow of ions within the reservoirs and thus irregularities in the delivery of active agent to the subject. Such difficulties in the use of highly viscous polymer solutions to fill porous substrates intended for the preparation of reservoirs may also be encountered when the fill matrix comprises sol-forming compounds, including those identified elsewhere herein, or a gel, such as a hydrogel.

In another possible approach to filling porous substrates for use in making reservoirs of devices described herein, and related devices, the highly viscous cellulosic polymer solution may be prepared without the active agent and/or electrolyte, as well as other compounds and/or excipients. The viscous polymer solution may then be dispensed, as above, into the porous substrate, followed by drying. In this approach, the dried polymer is then rehydrated by introducing into the porous substrate a solution of active agent and/or electrolyte, as well as other compound(s) and/or excipient(s). This approach, however, not only does not overcome the difficulties in the approach described above, but in fact adds further uncertainty regarding the efficiency and uniformity of rehydration of the dried polymer matrix.

Importantly, neither of these approaches is useful in producing suitable product via continuous web-based manufacturing operations. Such operations employ one or more webs of material, typically dispensed from one or more supply rolls and received on a take-up roll. Numerous manufacturing operations may be performed as the porous substrate web moves from the supply roll to the take-up roll. For example, various layers may be added to a web-type porous substrate, substances loaded into the substrate, and cuts may be made (e.g., die cutting, perforation, etc.). Continuous web-based manufacturing is the most promising approach to cost effective production of filled porous substrates for any use, such as drug-containing bandage material, and of passive and active devices, such as transdermal delivery devices. An important aspect of continuous web manufacturing is speed. Each of the manufacturing acts is typically performed while the web passes various pieces of equipment or work stations. Hence, the inability to quickly load a viscous material into a reservoir may adversely affect throughput of the manufacturing process.

Alternative to the approaches described above, further unexpectedly advantageous opportunities for filling porous reservoir substrates relate to temperature and concentration dependence of solubility of cellulosic polymers and to corresponding viscosity characteristics of the aqueous polymer solutions or suspensions.

As provided above, cellulosic polymers include, without limitation, hydroxypropyl cellulose, hydroxyethyl cellulose, hydroxypropylmethyl cellulose, and hydroxymethyl cellulose. Each of these is a cellulose ether derivative in which some of the hydroxyl groups of cellulose have been substituted. In hydroxypropyl cellulose (HPC), for example, some of the hydroxyl groups of cellulose have been hydroxypropylated, thus yielding cellulose molecules having —OCH₂CH(OH)CH₃ groups. In hydroxyethylated cellulose (HEC), some of the hydroxyl groups of cellulose have been hydroxyethylated, thus yielding cellulose molecules having —OCH₂CH₂OH. The other representative cellulose derivatives listed above correspond to hydroxypropylated methyl cellulose and hydroxymethylated cellulose, respectively.

While cellulose is not soluble in water, HPC becomes readily soluble at temperatures below those in a range of about 30-40° C.; as temperature increases through this range and higher, solubility decreases and aggregates begin to form. As discussed above, aqueous solutions of HPC are highly viscous and are thus difficult to dispense and to disperse throughout porous substrates intended for the production for example of pads, bandages, reservoirs and/or transdermal delivery devices. With increasing temperature of an HPC solution, however, as the solubility decreases and aggregates begin to form, the HPC solution, in certain embodiments, transforms into a suspension and the viscosity drops markedly. In certain such embodiments, with the increased temperature and the corresponding decrease in viscosity, the HPC suspension can be more readily dispensed into and dispersed throughout the porous substrate. In certain embodiments wherein the temperature is maintained at an appropriately elevated level, the HPC may be kept in suspension and the viscosity remains sufficiently low to enable dispensing and dispersal of the cellulosic polymer suspension containing active agent and/or electrolyte, as well as any further compounds and/or excipients. In such embodiments, as the temperature of the polymer suspension begins to drop, the polymer can again dissolve and the material thus remains dispersed throughout the porous substrate, again becoming a highly viscous medium for holding the various components within a reservoir.

In the process described above, as the temperature increases, the generation of uniform finely dispersed aggregates, thus resulting in a decrease in viscosity of the fluid, requires efficient heat transfer into and throughout the fluid and effective mixing of the fluid as it is heated.

In certain embodiments, as described herein, uniformity of highly viscous cellulosic polymer compositions dispersed throughout the porous substrate of various reservoirs in both passive and active delivery devices is preferred for consistent delivery of active agent to and through a biological interface. Further, in certain embodiments, in a device in which an active agent is delivered to a biological interface by application of an electromotive force or current, such as an iontophoresis device, uniformity of the polymer composition throughout the porous substrate of various electrolyte reservoirs is also preferred for consistent flow of electrolyte ions, which may thus aid in efficient operation of the device and delivery of the active agent. In some embodiments, such as those described above, in order to maintain uniformity of the highly viscous cellulosic polymer solution, it may be advantageous when raising the temperature to control the aggregation in such a manner that the aggregates so formed are finely divided aggregates, rather than larger aggregates or precipitates. In such embodiments, easier dissolution of such finely divided aggregates, when the temperature of the suspension is decreased, can provide a more uniform concentration of cellulosic polymer, active agent, and, as appropriate, electrolytes and/or other compounds and/or excipients. In this regard, it has been observed that, by incorporating hydroxyethyl cellulose (HEC) into solutions of hydroxypropyl cellulose (HPC) at appropriate ratios, the lower viscosity suspensions that result from increasing the temperature of the high viscosity liquid or sol comprises aggregates that are more finely divided than in the absence of HEC. In such embodiments, when the suspension so formed is dispensed into and dispersed throughout the porous substrate and the temperature is then allowed to drop, this finely divided HPC/HEC suspension re-dissolves and forms the highly viscous medium, which contains a more uniform distribution of the various components within the active or passive delivery devices than is obtained in the absence of HEC. Such an approach may also advantageously decrease the time it takes to load a porous substrate. In certain embodiments, such cellulosic polymer compositions may additionally, or alternatively, include hydroxypropyl methyl cellulose or methyl cellulose.

In view of the discussion above, it can be readily appreciated that it would be advantageous to have systems, methods and devices that are able to dispense solutions or suspensions of cellulosic polymers, having active agents and/or electrolytes, as well as other compounds or excipients, into and dispersed throughout porous substrates as discussed herein. It is particularly advantageous for such systems, methods and devices to be used in the manufacture of filled porous reservoir structures, particularly in continuous web-based manufacturing processes.

In this regard, systems and methods are disclosed herein for effective filling of porous reservoir structures such as those found in the reservoir elements of transdermal delivery devices for passive or active delivery of active agents to and/or through a biological interface of a subject. Although exemplified for filling reservoir elements of such devices, it will be readily apparent that the disclosed systems and methods can be used for effective filling of any type of porous substrate, for example pads, bandages, and the like. Further, although exemplified by embodiments utilizing continuous web-based processes for filling such devices, it will be readily apparent that the disclosed systems and methods can be used in filling porous structures manually or by other types of automated processes.

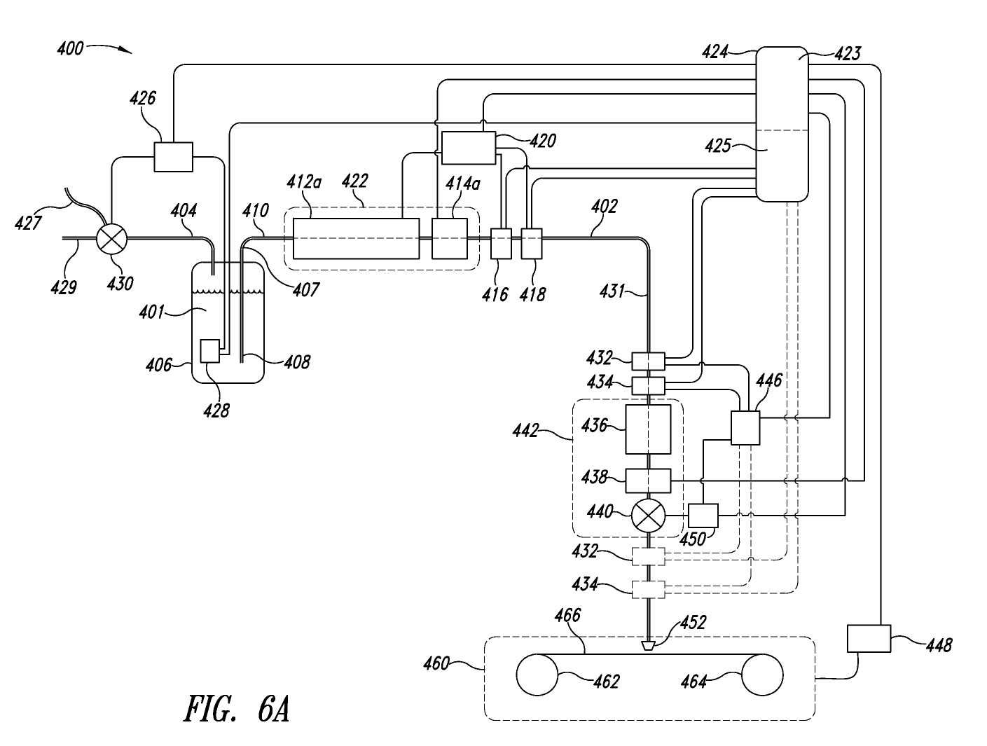

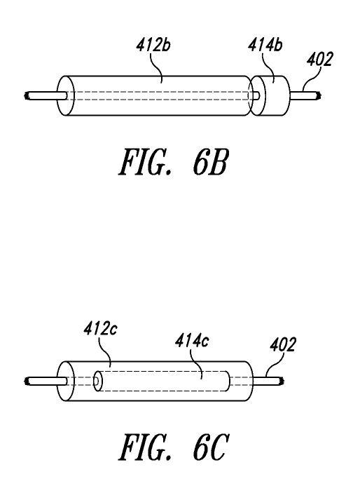

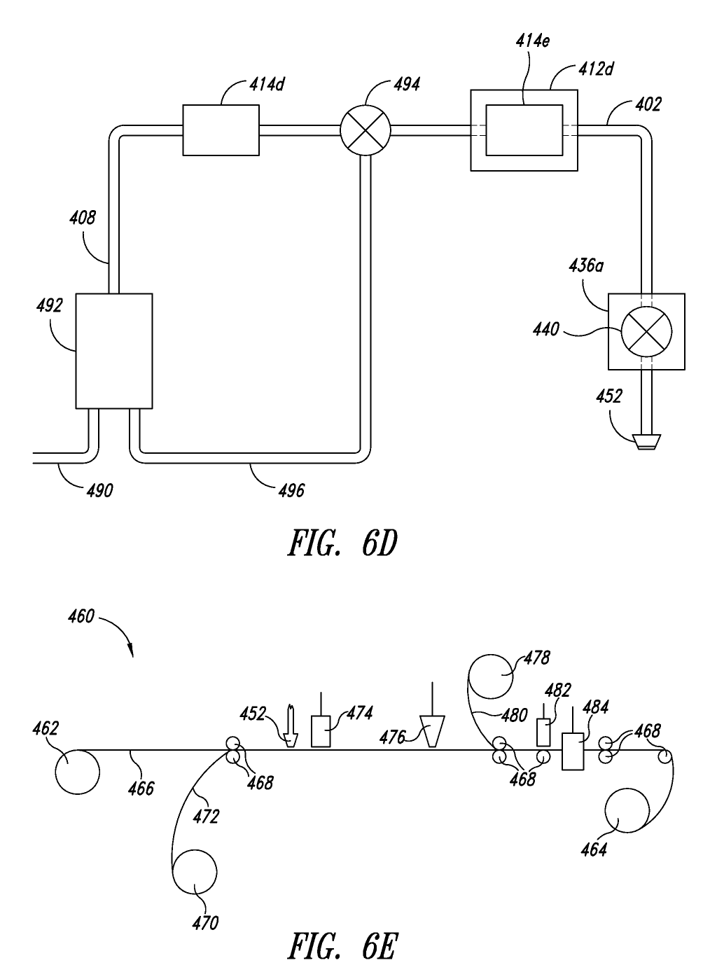

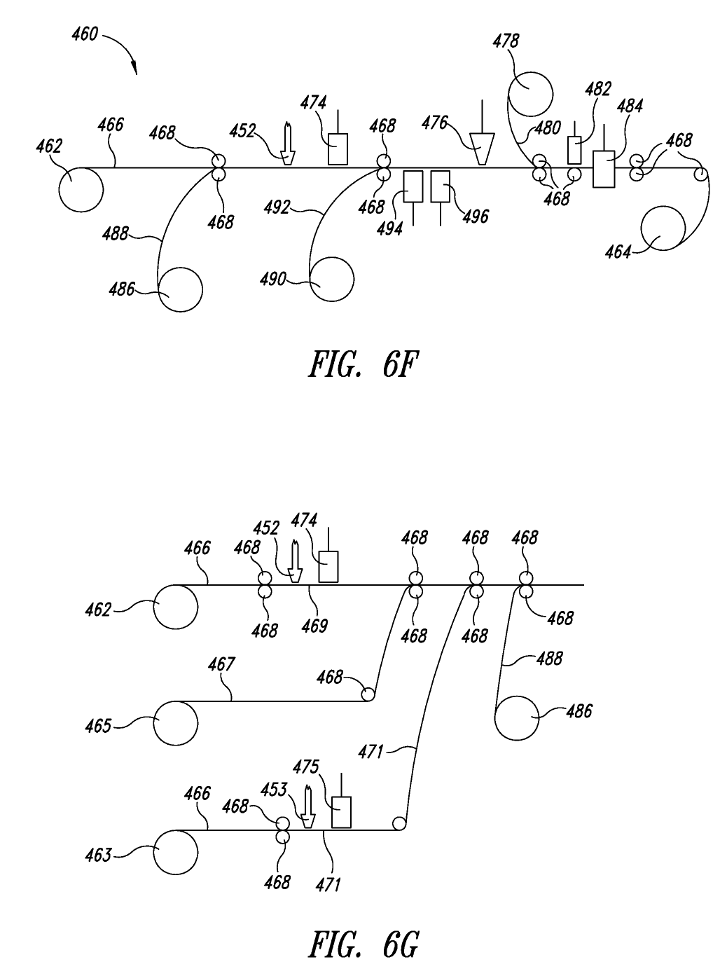

In one illustrated embodiment, FIG. 6A shows an exemplary system 400 suitable for dispensing a composition into a porous substrate. In particular, exemplary system 400 may be used to dispense a high viscosity HPC-based liquid or sol composition, or components thereof, into a porous substrate supplied by a continuous web-based process and suitable for use as a reservoir material. System 400 includes a conduit 402 having an inlet 408 and a dispensing outlet 452, conduit 402 further having a first portion 410 and a second portion 431, wherein first portion 410 is positioned between inlet 408 and second portion 431, and wherein second portion 431 is positioned between first portion 410 and dispensing outlet 452. System 400 further includes a dispensing reservoir 406 to hold a highly viscous liquid, a sol or a sol-forming composition 401, a first heater 412a, optionally a first mixer 414a, and a dispensing valve 440. Dispensing reservoir 406 further includes an inlet 404 and an outlet 407. Inlet 404 of dispensing reservoir 406 may be used, for example, to fill dispensing reservoir 406 with the highly viscous liquid, the sol or the sol-forming composition supplied via delivery tube 429. Inlet 404 may further be connected via pressure tubing 427 to a pressure source, e.g., pump, head (not shown) and used to apply pressure to the contents of dispensing reservoir 406 to drive the contents of dispensing reservoir 406 into and through conduit 402. Delivery of composition from composition inlet 429 or application of pressure from pressure inlet 427 via reservoir inlet 404 to dispensing reservoir 406 is selected by operation of inlet valve 430. Operation of inlet valve 430 is controlled by inlet valve controller 426. Operation of inlet valve controller 426 may be controlled by a signal from the output side 423 of master system controller 424. Upon receipt of an appropriate signal from master system controller 424, inlet valve 430 may be positioned to supply pressure, to supply composition, or to supply neither (i.e., an off position). Inlet controller 426 may, alternatively, operate in response to a signal from a reservoir level sensor 428. For example, a signal from reservoir level sensor 428 indicating that the composition level in the dispensing reservoir 406 is low may position inlet valve 430 to supply composition to re-fill dispensing reservoir 406.

In certain embodiments, as further discussed below, highly viscous liquid, sol or sol-forming composition 401 may be supplied directly to the conduit 402 via some type of metering pump (see, FIG. 6D), e.g., a positive displacement pump. Such a pump may controllably supply the highly viscous fluid or composition 401 to conduit 402 directly via inlet 408, thus taking the place of reservoir 406. Such a pump may be operated either manually or by a signal from the master system controller 424.

In exemplary system 400, first heater 412a and first mixer 414a are positioned at first portion 410 of the conduit. First heater 412a and first mixer 414a may optionally be included within a first heater/mixer unit 422. First heater 412a and first mixer 414a may exist within system 400, or within heater/mixer unit 422, as separate components or integrally associated with one another, as further described below (see, FIGS. 6B and 6C), first heater generically referenced as 412 and first heater generically referenced as 414. First mixer 414 may be a static or a dynamic mixer. In certain embodiments, as further discussed below, a mixer may not be necessary in systems wherein conduit 402 is narrow diameter capillary tubing. In the absence of a mixer, mixing may occur within such tubing simply by virtue of the flow of the fluid within narrow diameter of the tubing. Further, the narrow diameter of the tubing may allow for efficient heat exchange throughout the flowing composition 401. System 400, as illustrated, may include a first temperature controller 420 to regulate the temperature of first heater 412 and may optionally include a meter (not shown) to allow a human operator to monitor the temperature of first heater 412 and/or of the contents of conduit 402. Some embodiments of system 400 may employ a temperature sensor 416 (e.g., a thermocouple, a resistive temperature device, a thermistor, an infrared radiator, a bimetallic device, a liquid expansion device and/or a charged state device, etc.) and/or a pressure sensor 418 (an absolute or differential pressure transducer) to measure temperature and/or pressure, respectively, and provide appropriate signals to temperature controller 420 directly or to master system controller 424 (e.g., microcontroller, field programmable gate array or Application Specific Integrated Circuit), which in turn provides signals (raw or processed) to temperature controller 420. Master system controller 424 may have an input side 425, which receives signals, and an output side 423, which sends signals. Signals received at input side 425 of master system controller 424 may originate from various sensors within the system, including temperature sensor 416 and/or pressure sensor 418. Signals sent from output side 423 of master system controller 424 may be in response to signals received at input side 425 and/or may result from instructions provided by system software or firmware, or wired into system hardware, and/or may result from instructions provided manually by a human operator. Mixer 414, if a dynamic mixer, may be under the control of signals from master system controller 424.

In exemplary system 400, dispensing valve 440 is positioned at second portion 431 of conduit 402. Also positioned at second portion 431 of conduit 402 are a second heater 436 and, optionally, a second mixer 438. In the exemplary embodiment of FIG. 6A, second heater 436 is positioned before dispensing valve 440, and optional mixer 438 is positioned between second heater 436 and dispensing valve 440. At least two of dispensing valve 440, second heater 436 and second mixer 438 may optionally be included together, as separate components or integrally associated with one another, within a valve/heater/mixer unit 442. For example, a single apparatus may contain dispensing valve 440, second heater 436 and second mixer 438 (static or dynamic), all of which may be separately controllable. System 400 includes a dispensing valve controller 450 to regulate flow from dispensing outlet 452 of conduit 402 into a porous substrate 466. Porous substrate 466 may be positioned to receive composition 401 from dispensing outlet 452 of system 400 using a web-based manufacturing system 460 to supply porous substrate 466 by a continuous web-based process, as illustrated schematically. System 400 further includes a second temperature controller 446 for regulating the temperature of second heater 436 and may optionally include a meter (not shown) to allow a human operator to monitor the temperature of second heater 436 and/or the contents of conduit 402. Some embodiments of system 400 may employ a second temperature sensor 432 (e.g., a thermocouple, a resistive temperature device, a thermistor, an infrared radiator, a bimetallic device, a liquid expansion device and/or a charged state device, etc.) and/or a second pressure sensor 434 (an absolute or differential pressure transducer) to measure temperature and/or pressure, respectively, and provide appropriate signals to temperature controller 446 directly or to master system controller 424 (e.g., microcontroller, field programmable gate array or Application Specific Integrated Circuit), which in turn provides signals (raw or processed) to temperature controller 446. Dispensing valve controller 450 operates in response to signals received from master system controller 424. Such signals may result from input to master system controller 424 from various system sensors, in particular, second temperature sensor 432 and/or second pressure sensor 434. In certain embodiments, signals may be received by dispensing valve controller 450 from second temperature controller 446, which may be indicative of, for example, input to second temperature controller 446 from second temperature sensor 432 and/or second pressure sensor 434.

While the embodiment of dispensing system 400 illustrated in FIG. 6A shows elements of the system in particular positions, it would be readily understood by one of skill in the relevant art that certain illustrated elements may be positioned differently, such as to provide certain advantages in the design or operation of the system. As illustrated, for example, second temperature sensor 432 and second pressure sensor 434 may be advantageously positioned between dispensing valve 440 and dispensing outlet 452 of conduit 402 as an indicator of the temperature and/or viscosity of the composition prior to dispensing into porous substrate 466.

In the exemplary dispensing system 400 of FIG. 6A, composition 401 is dispensed from dispensing outlet 452 of conduit 402 into porous substrate 466 with a continuous web-based process. Within this simplified schematic diagram of a manufacturing system 460, the porous substrate 466 is provided from supply roller 462 and taken up by take-up roller 464. Operation of manufacturing system 460 is under control of a manufacturing system controller 448, which may be under the control of master system controller 424. Alternatively, manufacturing system controller 448 may operate manufacturing system 460 independent of control by master system controller 424. Manufacturing system 460 may include one or more further aspects, some of which are discussed below.

Flow of composition 401 through conduit 402 occurs by activation of inlet valve 430 to pressurize reservoir 406 via inlet valve inlet 427, which is connected to a pressure source (not shown). The pressure source may be pressurized gas, any of a variety of pumps, or simply an amount of head. The valve 430 may be operated automatically under control of master system controller 424, or manually, as appropriate.

Certain embodiments of FIG. 6A illustrated in FIGS. 6B and 6C show a mixer 414 that mixes heated contents of conduit 402 during transport of the contents through conduit 402. In FIG. 6B, mixer 414b is shown to be separate from heater 412b. In such embodiment, mixer 414b serves to mix the contents of conduit 402 following heating of the contents by heater 412b and transport of the heated contents via the portion of the conduit that exits from heater 412b. In the embodiment of FIG. 6C, mixer 414c is illustrated as an integral part of, or contained within, heater 412c. In such an embodiment, mixer 414c mixes the contents of conduit 402 during heating of the contents by heater 412c and transport of the heated contents via the portion of the conduit that is contained within heater 412c. In embodiments such as those illustrated in FIGS. 6B and 6C, during heating and transport of the highly viscous liquid, the sol or the sol-forming components and other components of composition 401, mixer 414b, 414c generates a well-mixed, uniform solution and/or suspension of the components.

In various embodiments, mixer 414 may be any of a variety of static mixing devices, including those having internal structures that mix composition 401 during passage of composition 401 through mixer 414. In certain other embodiments, mixer 414 may be any of a variety of dynamic mixers, including those having internal structures that may be controllably moved or vibrated to mix composition 401 during flow through mixer 414. Mixing by a dynamic mixing device may be controlled manually or automatically.

FIG. 6D shows a metering pump 492 to supply the highly viscous liquid, the sol, or the sol-generating composition to conduit 402 and two mixers 414d, 414e to mix the contents of conduit 402 during transport of the contents through conduit 402. The metering pump 492 may serve as an alternative to the combination of pressure inlet 427 and dispensing reservoir 406 of FIG. 6A for supplying the highly viscous liquid, sol, or sol-generating composition to inlet 408 of conduit 402. The highly viscous liquid, sol, or sol-generating composition is provided to metering pump 492 via inlet 490 and is then supplied by metering pump 492 via inlet 408 to conduit 402. The contents of conduit 402 flow from inlet 408 via conduit 402 through mixer 414d. Mixer 414d may be positioned within the system 400 to mix the contents of conduit 402 before heating the contents of conduit 402. The mixed contents of conduit 402 flow through optional valve 494. Valve 494, if present, may be set to return some or all of the mixed contents of conduit 402 via conduit 496 to metering pump 492, for example to be delivered again to mixer 414d for further mixing. When valve 494 is absent or is not configured or set to deliver the mixed contents of conduit 402 from mixer 414d back to metering pump 492, the mixed contents of conduit 402 are delivered from mixer 414d to mixer 414e. In certain embodiments, mixer 414e may be heated by heater 412d. For example, in certain such embodiments, mixer 414e may be positioned within a heater, such as a heating block. In other embodiments, heater 412d may be positioned to heat the contents of conduit 402 before or after mixing of the contents of conduit 402 by mixer 414e. In any such embodiments, the heated, mixed contents of conduit 402 flow to dispensing valve 440. Valve 440 may be heated. For example, valve 440 may be embedded in a second heater 436a, such as a heating block. Heater 436a may be temperature controlled, for example by control systems such as those shown schematically in FIG. 6A. Any reference herein to heater 436 or second heater 436 will be understood to include second heater 436a unless the context clearly indicates otherwise. The dispensing valve 440 may be set to dispense the mixed, heated contents of conduit 402 via dispensing outlet 452. In certain embodiments of the dispensing system shown in FIG. 6D, mixer 414d may be a static mixer and mixer 414e may be a dynamic mixer. In certain embodiments of FIG. 6D, control of the system and operation may occur by certain of the control processes suggested in FIG. 6A. For example, pressure monitors and/or pressure transducers may be positioned to monitor temperature and/or pressure of the composition 401 at various locations within conduit 402.

A further embodiment of FIG. 6A is illustrated in FIG. 6E.

In certain embodiments of the systems described herein, as exemplified above, mixer(s) may not be necessary. As discussed above, the mixing function of one or more of the mixers may alternatively be provided by use of a narrow diameter capillary tube as the conduit 402. During fluid flow through such a tube, effective mixing of the fluid and efficient heat exchange from the wall to the fluid and within the fluid may occur because of the narrow diameter of the tube.

Mixing, whether by use of a static or dynamic mixer or simply by flow of the fluid within a narrow diameter tubing, is necessary to provide efficient exchange of heat from the heater throughout the fluid via the walls of the conduit. The combination of mixing with efficient heat exchange advantageously provides a uniform composition with finely dispersed aggregates for dispensing. Effective mixing limits development of temperature gradients within the fluid, particularly areas of excessive heat against the inner walls of the conduit, which may lead to formation of aggregates of undesirable sizes or characteristics.

Mixers, such as those exemplified above as 414, 438, or within heater/mixer units 422, 442, may mix statically, dynamically, or by flow within a narrow diameter capillary tube. A mixer or a heater/mixer unit may include mixer components to provide a combination of different modes of mixing. For example, a mixer may include two components, one a static mixer and the other a dynamic mixer. In such a device, the fluid may enter the static mixer first, followed by the dynamic mixer. Alternatively, the fluid may enter the dynamic mixer first, followed by the static mixer. Further, mixing may include combining a static mixer and/or a dynamic mixer with fluid mixing via flow through capillary tubing.

In further illustrated embodiments of FIG. 6A, FIGS. 6E, 6F and 6G show in more detail particular aspects of web-based manufacturing system 460 particularly suitable for implementation of a continuous web-based process for filling porous substrates with high-viscosity HPC-containing compositions and producing transdermal delivery devices or components thereof.

FIG. 6E shows an embodiment of a continuous web-based manufacturing system 460 that may be particularly suited for production of a passive transdermal delivery device or porous reservoir components thereof. Web-type porous substrate 466 is provided from a web supply roll 462. When producing a passive transdermal delivery device, rather than simply filled porous reservoir components, a backing supply roll 470 may supply a backing material 472 for application to the distal surface of web-based porous substrate 466, that is, the surface of the porous substrate that in use is opposite to that intended to contact a skin of a subject. Backing material 472 may be any material suitable for use on a side of a transdermal delivery device that is not intended to contact a biological interface, such as a skin. For example, such a backing material may be any of a variety of inert, flexible, polymeric materials that can protect the distal surface of the device reservoir during use. The proximal surface of backing material 472 may include an inert adhesive layer (not shown), which may fix the proximal surface of backing material to the distal surface of porous substrate 466. Porous substrate 466 and backing material 472 are fed through rollers (e.g., pinch rollers) 468 to bring the two materials into contact. Composition 401 is applied to a surface of porous substrate 466 from dispensing outlet 462. When the distal surface of porous substrate 466 is bonded to backing material 472, composition 401 is applied to the surface of porous substrate 466 opposite to that having backing material 472. When porous substrate 466 does not include backing material 472, composition 401 may be applied to either surface of porous substrate 466, that is, either surface may act as the proximal surface. In certain embodiments, following filling of porous substrate 466 with composition 401, it may be advantageous to provide a flow of air, or some other gas, toward the surface of porous substrate 466 from a device (e.g., blower, fan, nozzle, etc.) 474 designed for that purpose. For example, the air so supplied may be dehumidified and/or at an elevated temperature to facilitate drying, either partially or completely, of composition 401 in porous substrate 466. Alternatively, the air so supplied may be humidified to prevent drying of composition 401 in porous substrate 466. In either of these embodiments, the air may be at ambient temperature. Although the embodiment in FIG. 6E shows dispensing outlet 452 and device 474 positioned after the application of the optional backing material 472, it would be readily appreciated by one of skill in the relevant art that dispensing outlet 452 and device 474 could be positioned prior to application of backing material 472. It would also be readily apparent to one of skill in the relevant art that alternative approaches could be taken to dry or humidify porous substrate 466 after application of composition 401. For example, drying could be accomplished by lengthening the path taken by porous substrate 466, thereby increasing the time that the porous substrate is exposed to the ambient temperature.

In the embodiment of system 460 in FIG. 6E, a release liner 480 may be applied to the proximal surface of filled porous substrate 466, that is, to the surface that is intended to be applied to the skin of a subject. In such embodiments, an adhesive dispenser 476 may be positioned to dispense pressure-sensitive adhesive to the proximal surface of filled porous substrate 466. Release liner 480 is supplied from a release liner roll 478 and is fed, together with filled porous substrate 466, between rollers (e.g., pinch or nip rollers) 468. Use of release liners and pressure-sensitive adhesives in this manner are known in the art and are not further discussed here. In use, when release liner 480 is removed from a transdermal delivery device produced in this manner, that is, prior to application of the device to a skin, the pressure-sensitive adhesive remains associated with the release liner, thus exposing filled porous substrate 466 to the skin.

In the embodiment of manufacturing supply system 460 shown in

FIG. 6E, the filled web-type porous substrate 466, having one or both a backing material 472 and a release liner 480 associated therewith, may be further continuously processed to produce a passive transdermal delivery device. For example, the web-type structure so produced may pass through a cutter 482, such as a die cutter, a slicer and/or a perforator, to produce a structure having a form appropriate for use as a particular transdermal delivery device. One or more pick-and-place devices 484 may supply an appropriate housing for the form of the unit produced by cutting device 482. Production of the transdermal delivery device, having the filled porous structure into the housing, may be completed, for example, by passage through rollers 468 so that the elements of the device are stably associated. Transdermal delivery devices so produced may be removed from the continuous production line and packaged by any of a variety of methods known in the art, and the remaining unused web structure may be taken up by the take-up roll 464. For example, the resulting transdermal delivery devices may be hermetically sealed in foil pouches, which may be formed as part of the continuous web manufacturing operation, using foil supply rolls and an adhesive or heat sealing operation. The operation of various elements of the web-based manufacturing system 460 may be controlled by a manufacturing system controller 448 (see, FIG. 6A), which may be controlled by master system controller 424 or may operate independently of other input. Elements of system 460 that may be controlled by manufacturing system controller 448 include motors (not shown) driving various rolls, device 474, adhesive dispenser 476, cutter 482 and pick-and-place device 484.

FIG. 6F shows a web-based filled porous substrate manufacturing system 460 that may be particularly suited for production of an active transdermal delivery device, such as an iontophoresis device, or porous reservoir components thereof. Web-type porous substrate 466 is provided from the web supply roll 462. When producing an active transdermal delivery device, rather than simply filled porous reservoir components, an insulative substrate roll 486 may optionally supply an electrically insulative substrate 488 to the distal surface of the web-type porous substrate 466. Electrically insulative substrate 488 may be designed to advantageously allow passage of electrical signals at certain defined portions of electrically insulative substrate 488, thereby providing in a device electrical continuity between the two sides of the insulative substrate 488 at pre-defined positions within the device. The proximal surface of electrically insulative substrate 488 may include a biologically inert adhesive layer (not shown), which may fix the proximal surface of electrically insulative substrate 488 to the distal surface of porous substrate 466. When electrical conductivity is required between two sides of insulative substrate 488 at pre-defined positions during operation of an active transdermal delivery device, the adhesive layer should not interfere with the electrical conductivity. Porous substrate 466 and electrically insulative substrate 488 are fed through rollers 468 to bring the two materials into contact. Composition 401 is then supplied to the proximal surface of porous substrate 466 from dispensing outlet 462. As discussed above related to FIG. 6E, it may be advantageous, in certain embodiments, following application of composition 401 to porous substrate 466, to provide a flow of air, or some other gas, toward the surface of porous substrate 466 from a device 474 to dry or humidify the composition 401. Although the embodiment in FIG. 6F shows dispensing outlet 452 and optional device 474 positioned after the position at which electrically insulative substrate 488 may be applied to the distal surface of porous layer 466, dispensing outlet 452 and device 474 could be positioned between porous substrate supply roll 462 and the position at which electrically insulative substrate 488 is applied. Porous substrate 466 may thus be filled with composition 401 before application of electrically insulative substrate 488.

In the embodiment of system 460 shown in FIG. 6F, when used in the production of an active transdermal delivery device, an electrode layer 492 may optionally be supplied from an electrode roll 490 and applied to the distal side of electrically insulative substrate 488. Electrode layer 492 may include a biologically inert adhesive layer (not shown) on its proximal surface, that is, on the surface that contacts the distal side of electrically insulative substrate 488. Electrode layer 492 and porous substrate 466 having attached electrically insulative substrate 488 are fed through rollers 468 to bring the two into contact.

Manufacturing system 460 in FIG. 6F may further advantageously include, for the purpose of producing active transdermal delivery devices, or active or counter electrode assemblies thereof, one or more pick-and-place device(s) 494 to provide additional circuit elements necessary for operation of the transdermal delivery device and/or a print head 496 to provide certain markings useful in use of and/or in identification of the devices or assemblies. Pick-and-place device 494 may, for example, provide certain printed circuit elements and/or a battery element. While pick-and-place device 494 and print head 496 are shown in particular locations in FIG. 6F, it would be readily apparent to one of skill in the relevant art that these elements could be advantageously placed elsewhere in manufacturing system 460.

In the embodiment of system 460 shown in FIG. 6F, when system 460 is used in the production of an active transdermal delivery device, or components or assemblies thereof, a release liner 480 may be applied to the proximal surface of filled porous substrate 466, that is, to the surface that is intended to be applied to the skin of a subject. In such embodiments, an adhesive dispenser 476 is positioned to dispense pressure-sensitive adhesive to the proximal surface of filled porous substrate 466. Release liner 480 is supplied from a release liner roll 478 and, together with filled porous substrate 466 and one or more of electrically insulative substrate 488, electrode layer 492, and additional circuit elements, is fed between rollers 468. During use, when release liner 480 is removed from a transdermal delivery device or electrode assemblies produced with system 460 as described herein, the pressure-sensitive adhesive remains associated with the release liner, thus exposing filled porous reservoir 466 to the skin.

To produce a structure appropriate for use as an active electrode assembly or a counter electrode assembly in an active transdermal delivery device, the form and extent of the electrode material 492 and the design of the electrically insulative substrate 488 may be such as to provide the electrical conductivity required for electrical continuity when the device is put into use.

FIG. 6G shows a further portion of web-based porous substrate manufacturing systems 460 of FIG. 6F and described above. In certain embodiments of an active transdermal delivery device, active electrode assemblies may comprise at least an active agent reservoir and an electrolyte reservoir. Likewise, in certain embodiments of an active transdermal delivery device, counter electrode assemblies may comprise two electrolyte reservoirs. FIG. 6G shows a portion of the web-based manufacturing system 460 of FIG. 6F that may be used to produce devices or electrode assemblies having two reservoirs proximate to one another. While it is understood that each of the two web-type porous substrates may be filled with any combination of an active agent or an electrolyte, the following discussion is directed for simplicity to production of devices in which one porous substrate comprises an active agent and the other comprises an electrolyte. Web-type porous substrate 466 supplied from web supply roll 462 passes between rollers 468 and is positioned to receive active agent-containing composition from dispensing outlet 452, thereby forming active agent-filled porous substrate 469. Active agent-filled porous substrate 469 moves to a position at which air flow may optionally be provided by device 474. A membrane 467 supplied from membrane roll 465 passes between rollers 468 and is positioned against the distal surface of active agent-filled porous substrate 469. Web-type porous substrate 466 supplied from web supply roll 463 passes between rollers 468 and is positioned to receive electrolyte-containing composition from dispensing outlet 453, thereby forming electrolyte-filled porous substrate 471. Electrolyte-filled porous substrate 471 moves to a position at which air flow may optionally be provided by device 475 and then between rollers 468 to be positioned against the distal surface of membrane 467. The composite web-based porous substrate thus produced, comprising active agent-filled and electrolyte-filled porous substrates 469, 471 with a membrane 467 positioned therebetween, moves on through manufacturing system 460 to form active transdermal delivery devices, or components thereof, as described above. Accordingly, the composite structure moves further through rollers 468 where electrically insulative substrate 488, supplied from substrate roll 486, is positioned against the distal surface of the electrolyte-filled porous substrate 471 component of the composite structure, and so on. In certain embodiments, membrane 467 may be a semi-permeable membrane or an ion-exchange membrane. In certain other embodiments, the membrane 467 may be an impermeable membrane, which can be removed prior to operation of the device to allow movement of electrolyte ions between an electrolyte-filled porous substrate reservoir and an active agent-filled porous substrate reservoir.

While the embodiments of web-based porous substrate manufacturing systems 460 or details thereof illustrated in FIGS. 6E-6G are described as being particularly suitable for production of a passive transdermal delivery device or porous reservoir components thereof, it would be readily understood by one of skill in the relevant art that such systems may be advantageously used for the production of various other types of filled porous substrates, such as pads, bandages, and the like.

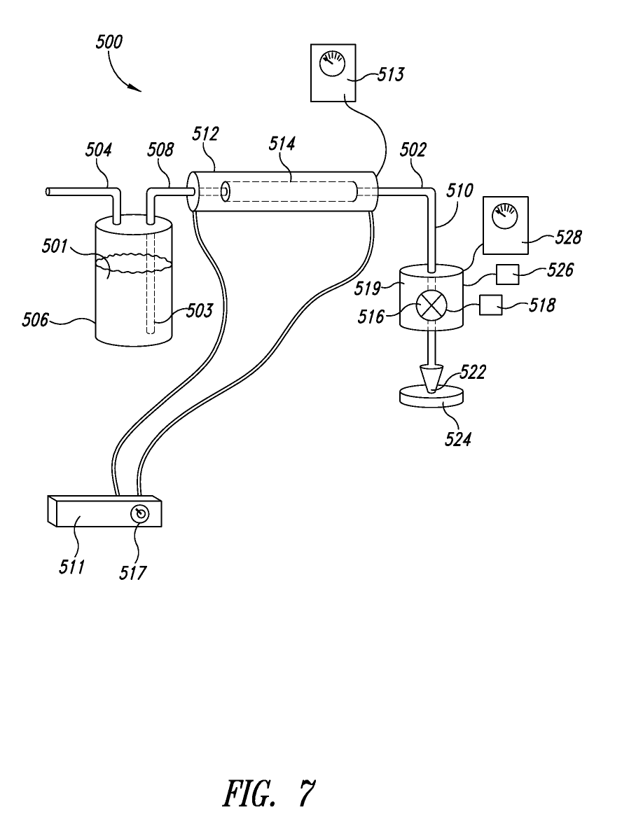

In a particular embodiment of the systems and methods described herein, FIG. 7 shows a manually operated system 500 to dispense a high viscosity liquid or sol composition, or composition of sol-forming components, into a porous substrate reservoir 524 (e.g., a transdermal device reservoir). A dispensing reservoir 506 contains composition 501. Composition 501 may be supplied to dispensing reservoir 506 via reservoir inlet 504. Composition 501 moves from the dispensing reservoir 506 to a dispensing outlet 522 via a conduit 502. Pressure may be applied to reservoir inlet 504, for example, by use of pressurized gas or a pump, to move composition 501 through system 500 via conduit 502. Conduit 502 has a first portion 508 and a second portion 510. First portion 508 is positioned between a first end 503 and second portion 510. Second portion 510 is positioned between first portion 508 and dispensing outlet 522. First portion 508 of conduit 502 includes a heat exchanger 512 with an enclosed or integral mixer 514. A circulating water bath 511 with a temperature controller 517 provides heated water to the heat exchanger 512. Water provided by circulating water bath 511 is set using temperature controller 517 to a temperature appropriate to maintain the temperature of composition 501 with heat exchanger 512 at a first temperature selected by a human operator of the system 500. The temperature within heat exchanger 512 may be measured using a temperature sensor (not shown) within heat exchanger 512 and displayed on a meter 513. The human operator may adjust temperature controller 517 based on the temperature displayed on meter 513. Second portion 510 of conduit 502 includes a dispensing valve 516 to controllably dispense composition 501 from dispensing outlet 522 of system 500 into porous substrate reservoir 524. Dispensing valve 522 may be controlled by a dispensing valve controller 518. The human operator may operate dispensing valve controller 518 and/or may adjust the pressure applied to reservoir inlet 504 to control the rate at which composition 501 is dispensed from dispensing outlet 522 into porous substrate reservoir 524. The system 500 may further include a heater 519 proximate to second portion 510 of conduit 502 to heat the contents of second portion 510 of conduit 502 to a second temperature. Heater 519 may be positioned at the position of dispensing valve 516. In particular embodiments, heater 519 may surround dispensing valve 516. In other embodiments, dispensing valve 516 and heater 519 may be a single unit, for example, a dispensing valve unit which incorporates a heating element. In yet other embodiments, heater 519 may be positioned between first portion 508 of conduit 502 and dispensing valve 516 or between dispensing valve 516 and dispensing valve outlet 522. Heater 519 may be controlled by a second temperature controller 526 to maintain the contents of second portion 510 of conduit 502 at the second temperature. In certain embodiments, in preparation for dispensing composition 501, it may be particularly advantageous to maintain the second temperature at a level less than that of the first temperature. The temperature within heater 519 may be measured using a temperature sensor (not shown) within heater 519 and displayed on a meter 528. The human operator may adjust temperature controller 526 based on the temperature displayed on the meter 528. In certain embodiments, temperature controller 526 may also be communicatively coupled to control dispensing valve 516. In FIG. 7, porous substrate reservoir 524 may be positioned within a housing of a transdermal delivery device before dispensing of composition 501 into porous substrate reservoir 524.

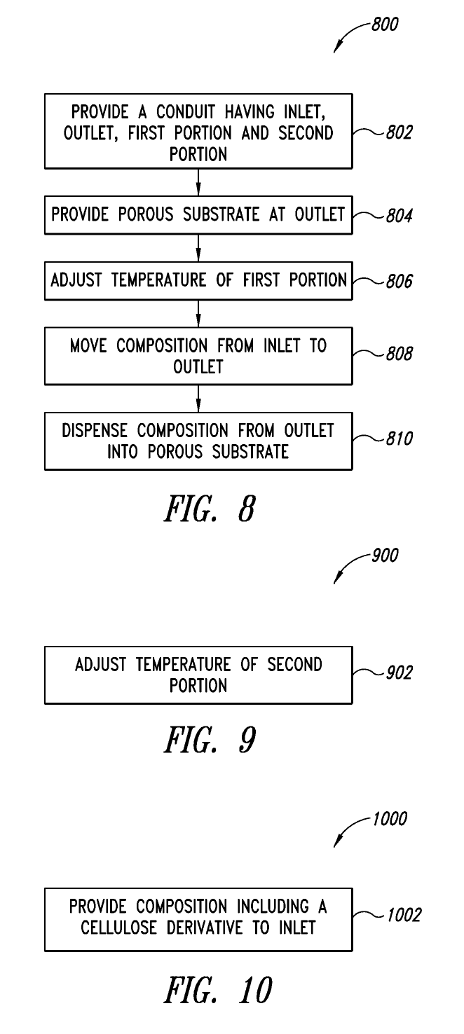

FIG. 8 shows, in one illustrated embodiment, a method 800 to use system 400 of FIGS. 6A-6G to dispense cellulose derivative-containing composition 401 into porous substrate 466, e.g., by a web-based manufacturing process. Method 800 may alternatively correspond to operation of system 500 of FIG. 7.

At 802, system 400 includes conduit 402 having inlet 408, dispensing outlet 452, first portion 410 and second portion 431. Conduit 402 provides a pathway for composition 401 from dispensing reservoir 406 or metering pump 492 via inlet 408, first portion 410, and second portion 431, to dispensing outlet 452. Alternatively, for system 500 in FIG. 7, system 500 includes conduit 502 having inlet 503, dispensing outlet 522, first portion 508 and second portion 510, which provides a pathway from dispensing reservoir 506 to dispensing outlet 522.

At 804, a web-based porous substrate 466 is provided at dispensing outlet 452 of conduit 402. Alternatively, in FIG. 7, a porous substrate reservoir 524 is provided at dispensing outlet 522 of the conduit.

At 806, first heater 412, located proximate to first portion 410 of conduit 402, is adjusted to a first temperature. First heater 412, so adjusted, heats at least a segment of the conduit and/or the contents of the conduit to the first temperature. First heater 412 is controlled by first temperature controller 420, which responds to signals from first temperature sensor 416 and/or first pressure sensor 418 and/or to signals from master system controller 424. Alternatively, in FIG. 7, first heater 512 heats at least a segment of first portion 508 of conduit 502 and/or its contents. First heater 512 in FIG. 7 is manually controlled by a human operator. The operator adjusts the first temperature via first temperature controller 517, which controls the temperature of circulating water bath 511. The operator can monitor the first temperature of first heater 512 by observing first temperature meter 513.

At 808, the composition moves from inlet 408 of conduit 402 to dispensing outlet 452. Inlet valve 430 is positioned to allow pressure from pressure inlet 427 to pressurized dispensing reservoir 406, or metering pump 492 is operated, and dispensing valve 440 is positioned to allow flow of composition 401 through conduit 402 from inlet 408 to outlet 452. Alternatively, for system 500 in FIG. 7, composition 502 moves from inlet 503 to dispensing outlet 522. Pressure is provided to pressurized dispensing reservoir 506 by connection of reservoir inlet 504 to a pressure source by the operator.

At 810, composition 401 is dispensed from dispensing outlet 452 into porous substrate 466, which may be a web-based porous substrate. Alternatively, for system 500 in FIG. 7, composition 501 is dispensed from outlet 522 into porous substrate reservoir 524.

FIG. 9 shows, in one illustrated embodiment of method 800 of FIG. 8, a method 900 to used system 400 of FIGS. 6A-6G to dispense cellulose derivative-containing composition 401 into porous substrate 466. As with method 800, method 900 may alternatively correspond to operation of system 500 of FIG. 7.

At 902, system 400 includes a second heater 436, located proximate to second portion 431 of conduit 402. Second heater 436 is adjusted to a second temperature and heats at least a segment of conduit 402 and/or the contents of conduit 402 to the second temperature. Second heater 436 is controlled by second temperature controller 446, which responds to signals from second temperature sensor 432 and/or second pressure sensor 434 and/or to signals from master system controller 424. Alternatively, in FIG. 7, second heater 519 heats at least a segment of second portion 510 of conduit 502 and/or its contents. As illustrated in FIG. 7, second heater 519 heats second portion 510 of conduit 502 proximate to dispensing valve 516, including within dispensing valve 516. For example, second heater 519 may be an integral part of dispensing valve 516. Second heater 519, as illustrated in FIG. 7, is manually controlled by the operator. The operator adjusts the second temperature via second temperature controller 526. The operator can monitor the second temperature of second heater 519 by observing second temperature meter 528.

FIG. 10 shows, in one illustrated embodiment of method 800 of FIG. 8, a method 1000 to use system 400 of FIGS. 6A-6G to dispense cellulose derivative-containing composition 401 into porous substrate 466. As with method 800, method 1000 may alternatively correspond to operation of system 500 of FIG. 7.

At 1002, system 400 includes within dispensing reservoir 406 or within metering pump 492 a cellulose derivative-containing composition 401. In such embodiments, composition 401 may be placed in dispensing reservoir 406 or metering pump 492 at any time before activation of system 400 to move composition 401 from inlet 408 to dispensing outlet 452. In certain embodiments, composition 401 is placed in dispensing reservoir 406 or metering pump 492 before the first temperature of first heater 412 is adjusted. In certain other embodiments, composition 401 is placed in dispensing reservoir 406 or metering pump 492 after the first temperature of first heater 412 is adjusted. In certain embodiments, when second heater 436 is also present, composition 401 is placed in dispensing reservoir 406 or metering pump 492 before both the first temperature of first heater 412 and the second temperature of second heater 436 are adjusted. In certain other embodiments, when second heater 436 is also present, composition 401 is placed in dispensing reservoir 406 or metering pump 492 after the first temperature of first heater 412 is adjusted but before the second temperature of second heater 436 is adjusted. In yet other embodiments, when second heater 436 is also present, composition 401 is placed in dispensing reservoir 406 or metering pump 492 after both the first temperature of first heater 412 and the second temperature of second heater 436 are adjusted. Similarly, for system 500 of FIG. 7, composition 501 may be placed in dispensing reservoir 506 at any time before activation of system 500 by the operator to move composition 501 from inlet 503 to dispensing outlet 522. As described above for system 400, composition 501 may be placed in dispensing reservoir 506 either before or after adjustment of the first temperature of first heater 512 and, when second heater 519 is present, the second temperature of second heater 519.

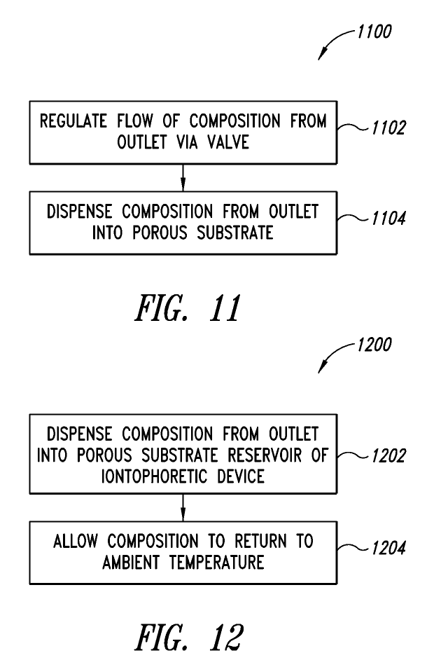

FIG. 11 shows, in one illustrated embodiment of method 800 of FIG. 8, a method 1100 to use system 400 of FIG. 6A-6G to dispense a cellulose derivative-containing composition 401 into porous substrate 466. As with method 800, method 1100 may alternatively correspond to operation of system 500 of FIG. 7.

At 1102, system 400 regulates flow of composition 401 through conduit 402 by controlling inlet valve 430 and/or metering pump 492 and/or dispensing valve 440. Either or both inlet valve 430 and dispensing valve 440 may be metering-type valves that can be adjusted to regulate flow, whether gas or fluid, through the valve. Inlet valve 430 may be adjusted to control pressure applied to dispensing reservoir 406, thereby controlling the rate at which composition 401 flows from dispensing reservoir 406 into inlet 408 and through conduit 402 to dispensing outlet 452. Inlet valve 430 is controlled by inlet valve controller 426, which, for the purpose of regulating flow through the system, is controlled by master system controller 424. In certain embodiments, metering pump 492 may be controlled by inlet valve controller 426 or directly by master system controller 424. Dispensing valve 440 may be adjusted to regulate flow of composition 401 from dispensing outlet 452 of conduit 402. Dispensing valve 440 is controlled by dispensing valve controller 450, the operation of which may be controlled by master system controller 424 and/or by second heater controller 446, when present. In certain embodiments, flow of the composition 401 through conduit 402 from inlet 408 to dispensing outlet 452 is regulated by inlet valve 430. In certain other embodiments, flow of composition 401 through conduit 402 from inlet 408 to dispensing outlet 452 is regulated by dispensing valve 440. In yet other embodiments, flow of composition through conduit 402 from inlet 408 to dispensing outlet 452 is regulated by both inlet valve 430 and dispensing valve 440.

Similarly, for manually operated system 500 of FIG. 7, dispensing valve 516 may be adjusted to regulate flow of composition 501 from dispensing outlet 522 of conduit 502. Dispensing valve 516 is manually controlled by the operator. The operator manually adjusts dispensing valve controller 516, thereby regulating the flow from dispensing outlet 522. In certain embodiments, the operator may, alternatively regulate the pressure applied at reservoir inlet 504 by adjusting a valve (not shown) between a pressure source or head and reservoir inlet 504, thereby regulating the flow of composition 501 from dispensing outlet 522.

At 1104, composition 401 is dispensed from dispensing outlet 452 into porous substrate 466 (e.g., a transdermal device reservoir). Porous substrate 466 may be any of a variety of forms of porous substrate appropriate for holding the composition 401. For example, porous substrate 466 may include various bulk porous substrates suitable for manufacturing processes. Such porous substrates may include web-based porous substrates, sheets, circles, rectangles or strips. Porous substrates may alternatively include porous substrates contained in various types of reservoirs suitable for use in devices, such as reservoirs that may be incorporated into passive or active transdermal delivery devices. Similarly, for system 500 of FIG. 7, composition 501 may be dispensed into any of a variety of porous substrates, including porous substrates contained in, or suitable to be placed in, reservoirs appropriate for passive or active transdermal delivery of active agents.

FIG. 12 shows, in one illustrated embodiment of method 800 of FIG. 8, a method 1200 to use system 400 of FIGS. 6A-6G to dispense a cellulose derivative-containing composition 401 into porous substrate 466. As with method 800, method 1200 may alternatively correspond to operation of system 500 of FIG. 7.

At 1202, composition 401 is dispensed from dispensing outlet 452 into a porous substrate 466 (e.g., an active transdermal delivery device reservoir, in particular an iontophoretic device reservoir). As above, such porous substrates may include web-based porous substrates, sheets, circles, rectangles or strips. Similarly, for system 500 of FIG. 7, composition 501 may be dispensed into any of a variety of porous substrates 524, including porous substrates contained in, or suitable to be placed in, reservoirs appropriate for active transdermal delivery, in particular, iontophoretic delivery, of active agents.

At 1204, dispensed composition 401 in porous substrate 466 is allowed to equilibrate to ambient temperature. As dispensed from system 400, composition 401 at an elevated temperature has a low viscosity compared to its viscosity at ambient temperature. Accordingly, composition 401 is dispensed into porous substrate 466 at low viscosity. Composition 401 fills porous substrate 466. As the temperature of composition 401 decreases, its viscosity increases. At ambient temperature, composition 401 is in the form of a highly viscous liquid, a sol, or a sol-like composition, within the porous substrate 466. Ambient temperature is preferably maintained at a level no higher than 30-35° C. Similarly, for system 500 of FIG. 7, composition 501 is dispensed into porous substrate 524 as a low viscosity composition at elevated temperature. Upon equilibration to ambient temperature, composition 501 takes the form of a highly viscous liquid, a sol or a sol-like composition, within the porous substrate 524.

The diagrammatic representations in FIGS. 6A-6G and 7 and the flow diagrams of FIGS. 8-11 are non-limiting examples of the systems described herein, and, as such, it would be readily apparent to one of skill in the relevant art that certain diagrammed or illustrated elements may be present, or not, and that certain elements, if present, may take forms or structures different from those diagrammed or illustrated. For example, flow of a composition of highly viscous liquid, sol or sol-forming components containing an active agent and/or an electrolyte and/or further compounds and/or expedients may result from applying pressurized gas or from any of a variety of pumps. Further, a heat source may be a circulating heated fluid, such as water from a circulating water bath, as illustrated, or any of a variety of electrical heating elements. As described above, mixing of a composition during transport through the conduit may be carried out by either static or dynamic mixing devices.

In certain embodiments of devices for transdermal delivery of active agents, a transdermal device reservoir may have a porous substrate that, by use of the systems and methods described herein, may be infiltrated uniformly by a composition that may contain an active agent and/or an electrolyte and/or further other compounds and/or excipients.

Systems and processes for dispensing high viscosity liquids, sols, or sol-forming compounds described herein are particularly advantageous for filling active agent and/or electrolyte reservoirs of passive and active transdermal delivery devices, including iontophoresis devices. Such systems and processes may be useful, for example, for filling porous substrate or transdermal device reservoirs of the devices exemplified above, but are not limited thereto.

### Examples

### EXAMPLE 1

### Hot Fill System

According to one embodiment of the description herein, a system for dispensing a composition (see, FIG. 6D) was made by providing a fluid connection between the outlet of a metering pump containing a composition and the inlet of a static mixer; a fluid connection between the outlet of the static mixer and the inlet of a feedback valve; a fluid connection between one outlet port of the feedback valve and the metering pump; a fluid connection between a second outlet port of the feedback valve and a dynamic mixer enclosed within a heater; a fluid connection between the dynamic mixer and a dispensing valve enclosed within heater; and a fluid connection between the outlet of the dispensing valve and a outlet tube to dispense a composition into a porous substrate to be filled with the composition. The temperatures of the dynamic mixer and the dispensing valve were maintained by operation of the respective heaters. Composition was dispensed into porous substrate by activation of a solenoid to allow composition to flow through the dispensing valve to the outlet tube. The metering pump was used to control the rate at which composition was dispensed from the outlet tubing into the porous substrate.

### Example 2

### Hot Fill System

According to one embodiment of the description herein, a system for dispensing a composition (see, FIG. 7) was made by providing a fluid connection between the outlet of a gas-pressurized reservoir (615DT Series, EFD, Inc., East Providence, R.I., USA) containing the composition and the inlet of a jacketed static mixer (Kenics, Chemineer, Dayton, Ohio, USA); a fluid connection between the outlet of the jacketed static mixer and the inlet of a dispensing valve (754V-SS, EFD, Inc., East Providence, R.I.); and tubing from the outlet of the dispensing valve to dispense the composition into a device to be filled with the composition. The Chemineer Kemics jacketed static mixer was a 36-element mixer having an internal diameter of ⅜ inch and a length of about 20 inches. The temperature of the mixer was maintained by circulating water at a pre-established temperature through the jacket using a 20-liter re-circulating water bath (Model 2095, Forma Scientific, Marietta, Ohio). The temperature within the jacket was monitored using a multimeter temperature monitor and probe (Supermeter® HHM290, Omega Engineering, Stamford, Conn.). The temperature of the dispensing valve was maintained by wrapping the valve with a heating element (Kapton insulated flexible heater, Model No. KH-303/5, Omega Engineering) and controlling the temperature with a controller (CSC32 Series, Omega Engineering). Actuating a dispensing valve controller (ValveMate 8000, EFD, Inc.) in combination with application of pressure to the contents of the reservoir at the beginning of the system was used to control the rate at which the composition was dispensed into the desired receptacle.

### Example 3

### Hot Fill Dispensing

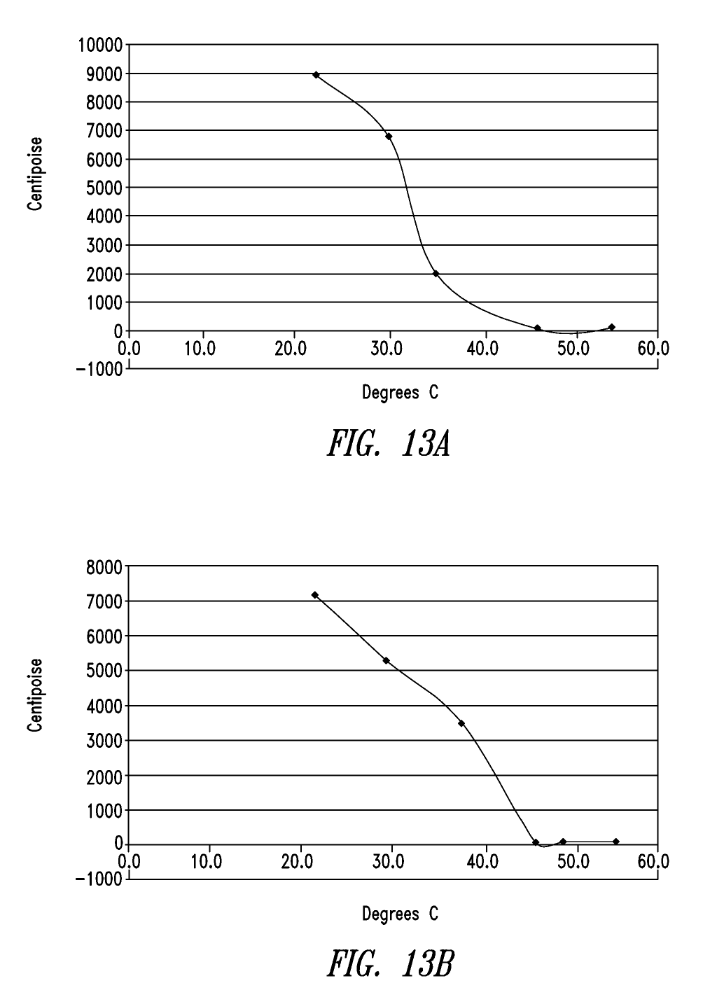

In particular embodiments of the disclosure, the system of Example 1 was used to dispense a counter electrode composition having 1.5% (w/w) hydroxypropyl cellulose and 0.5% (w/w) hydroxyethyl cellulose in an aqueous medium. Dispensing using the mixer jacket and the dispensing valve at various temperatures showed that dispensing was most reliable and consistent when the temperature was near or at the lower end of the viscosity-temperature plots shown in FIGS. 13A and 13B. Further, it was observed that the best results were obtained when the temperature of the dispensing valve was at or below the lowest temperature of the mixer jacket. After allowing the system to equilibrate under conditions with the mixer jacket at 52-53° C. and the dispensing valve at 50-51° C., the composition was dispensed six times with a 5-second delay between each. The results are provided in Table I and show consistency of delivery, with the average of the six deliveries being 506.7 mg, with a standard deviation of 6.1 and a percent coefficient of variation of 1.2%.

TABLE I

| Dispense | Dispense |
|---|---|
| Number | Weight (mg) |
| 1 | 495.2 |
| 2 | 506.0 |
| 3 | 511.4 |
| 4 | 507.6 |
| 5 | 512.2 |
| 6 | 507.7 |
| Average | 506.7 |
| Std. Dev. | 6.1 |
| % CV | 1.2% |

### Example 4

### Temperature Dependence of Viscosity of HPC/HEC Compositions

Active electrode compositions of active agent and counter electrode compositions were each prepared containing 1.5% (w/w) hydroxypropyl cellulose and 0.5% (w/w) hydroxyethyl cellulose. Each was heated to a specific temperature between 20° C. and 55° C., and viscosity was measured at that temperature. Viscosity (in centipoise) measured at each temperature (in ° C.) is shown for the counter electrode composition in FIG. 13A and for the active electrode active agent composition in FIG. 13B. For each, the viscosity reached its lowest point between 45° C. and 50° C.

### Example 5

### Evaluation of Active Electrode and Counter Electrode Formulations

In certain embodiments of the disclosure, counter electrode composition, placebo composition (epinephrine alone), and active agent electrode composition (lidocaine-epinephrine) were prepared as follows:

### Counter Electrode Composition

TABLE II

| | Concentration |
|---|---|
| Material | (% w/w) |
| Sodium chloride | 0.584 |
| Sodium phosphate, monobasic, anhydrous | 0.6 |
| Postassium sorbate | 0.135 |
| Hydroxyethyl cellulose | 0.5 |
| (Natrosol-250 M, Pharm. Grade) | |
| Hydroxylpropyl cellulose | 1.5 |
| (Klucel-MF, Pharm. Grade) | |
| Purified water | 96.681 |

### Placebo Composition

TABLE III

| | Concentration |
|---|---|
| Material | (% w/w) |
| Epinephrine bitartrate | 0.179 |
| Sodium chloride | 2.123 |
| Citric acid, anhydrous | 0.074 |
| EDTA, disodium, dihydrate | 0.15 |
| Sodium metabisulfite | 0.06 |
| Propylene glycol | 3.00 |
| Sorbic acid | 0.10 |
| Hydroxyethyl cellulose | 0.500 |
| (Natrosol-250 M, Pharm. Grade) | |
| Hydroxylpropyl cellulose | 1.500 |
| (Klucel-MF, Pharm. Grade) | |
| Purified water | 92.314 |

### Active Agent Electrode Composition

TABLE IV

| | Concentration |
|---|---|
| Material | (% w/w) |
| Lidocaine hydrochloride, monohydrate | 10.5 |
| Epinephrine bitartrate | 0.179 |
| Citric acid, anhydrous | 0.074 |
| EDTA, disodium, dihydrate | 0.15 |
| Sodium metabisulfite | 0.06 |
| Propylene glycol | 1.00 |
| Methylparaben | 0.18 |
| Propylparaben | 0.02 |
| Hydroxyethyl cellulose | 0.444 |
| (Natrosol-250 M, Pharm. Grade) | |
| Hydroxylpropyl cellulose | 1.556 |
| (Klucel-MF, Pharm. Grade) | |
| Purified water | 85.837 |

Differential scanning calorimetry (DSC) was performed on each composition using a Microcal VP-DSC instrument through a temperature range of 20-80° C. to measure transition endotherms. Transition temperatures and enthalpic change for the compositions were determined to be as follows: counter electrode composition, Tm=38.10±0.04° C., ΔH=5960±150 kcal/mole; placebo composition, Tm=37.15±0.04° C., ΔH=6330±130 kcal/mole; active agent composition (1^{st} scan), Tm=46.70±0.06° C., ΔH=6970±910 kcal/mole; and active agent composition (10^{th} scan), Tm=47.84±0.03° C., ΔH=4880±330 kcal/mole. DSC provides information concerning the composition that is complementary to that obtained from viscosity measurements. For example, the magnitude of enthalpic change may aid in selecting an appropriate composition.

### Example 6

### Evaluation of Hydroxypropyl Cellulose Formulations

In certain embodiments of the disclosure, formulations were prepared with different concentrations of hydroxypropyl cellulose as follows, in counter electrode, placebo, and active agent compositions:

TABLE V

| | Formulations, % w:w | | | | | |
|---|---|---|---|---|---|---|
| | 081210A | 081210B | 081211A | 081226A | 090106A | 090106B |
| | Counter electrode | | Placebo | Active agent | | |
| Material | composition | | comp | composition | | |
| HPC, Grade M | 1.50 | | | | | |
| HPC, Grade G | | 3.50 | | | | |
| HPC, Grade GF | | | | 4.00 | 3.25 | 1.50 |
| HPC, Grade MF | | | 1.50 | | | |
| HEC, 250M | | | 0.50 | | | |
| HEC, 250G | 0.50 | 0.50 | | 0.50 | 0.50 | 0.45 |
| Potassium | 0.135 | 0.135 | 0.135 | | | |
| sorbate | | | | | | |
| Sodium chloride | 0.585 | 0.585 | 2.123 | | | |
| Sodium phosphate, | 0.60 | 0.60 | | | | |
| monobasic, anhydrous | | | | | | |
| Poloxamer 188 | 0.05 | 0.05 | 0.05 | 0.05 | 0.05 | 0.05 |
| Propylene glycol | | | 2.00 | 2.02 | 2.00 | 2.00 |
| Lidocaine | | | | 10.50 | 10.47 | 10.50 |
| Epinephrine bitartrate | | | 0.179 | 0.179 | 0.179 | 0.179 |
| EDTA, disodium | | | 0.15 | 0.15 | 0.15 | 0.15 |
| Sodium metabisulfite | | | 0.06 | 0.06 | 0.06 | 0.06 |
| Citric acid, anhydrous | | | 0.074 | 0.075 | 0.073 | 0.073 |
| Methylparaben | | | | 0.18 | 0.18 | 0.18 |
| Propylparaben | | | | 0.02 | 0.02 | 0.02 |
| Purified water | 96.63 | 94.63 | 93.23 | 82.26 | 83.07 | 84.84 |

Differential scanning calorimetry (MicroCal Model VP-DSC) was performed on each composition to measure transition endotherms. Transition temperatures for the compositions were determined to be as follows:

TABLE VI

| | Formulations | | | | | |
|---|---|---|---|---|---|---|
| | 081210A | 081210B | 081211A | 081226A | 090106A | 09106B |
| Tm, ° C. | 41.21 ± 0.02 | 41.63 ± 0.03 | 41.21 ± 0.02 | 52.62 ± 0.03 | 52.11 ± 0.04 | 50.58 ± 0.5 |

For each composition viscosities (in centipoise) were determined through a range of temperatures using a Brookfield RVT viscometer. The spindle size used for each measurement varied with the temperature and thus the viscosity. The speed of rotations was 20 rpm throughout. The spindle numbers used in the viscometer ranged from a number 5 spindle at lowest temperature/highest viscosity to a number 1 spindle at highest temperature/lowest viscosity. The measured viscosities were as follows:

### Counter Electrode and Placebo Compositions

TABLE VII

| | Viscosity, in centipoise | | | | | |
|---|---|---|---|---|---|---|
| Temp., ° C. | 081210A | 081210B | 081211A | 081226A | 090106A | 090106B |
| 20.0 | | | | 11120 | | |
| 20.6 | | | | | 5840 | |
| 22.2 | 7300 | 3910 | 7720 | | | 9300 |
| 23.9 | | | | | 5280 | |
| 24.4 | | | | 9880 | | 8560 |
| 26.7 | 6140 | | | | 4800 | |
| 27.8 | | 3400 | 5000 | | | |
| 28.3 | | | | 8880 | | 7500 |
| 28.9 | | | | | 4420 | |
| 29.4 | 4600 | | | | | |
| 30.6 | | 3150 | | | | |
| 31.1 | | | | 8200 | | |
| 31.7 | 3950 | | | | 4150 | |
| 32.2 | | | | | | 4720 |
| 33.3 | 3080 | | 2050 | | | |
| 33.9 | | | | | 3800 | |
| 34.4 | | | | | | 3180 |
| 35.0 | | | | 7320 | | |
| 35.6 | 2000 | 1530 | 92.5 | | | |
| 36.7 | | | | | 3380 | |
| 37.8 | 366 | 1060 | | 6520 | | |
| 38.3 | | | | | | 2180 |
| 38.9 | 53.5 | 172 | | | 2990 | |
| 39.4 | | | | 5040 | | |
| 40.0 | 49 | 102 | | | | 1825 |
| 41.1 | | | | 4350 | | |
| 41.7 | | | | | 2370 | |
| 42.2 | 40 | 17 | | | | 355 |
| 43.3 | | | 41.5 | | | |
| 43.9 | | | | 3600 | 1825 | |
| 44.4 | | | | | | 100 |
| 46.1 | | | | | 860 | |
| 47.8 | | | | 1350 | | 37 |
| 48.3 | | | | | 332 | |
| 48.9 | 30.5 | 11.5 | 35 | | | |
| 50.0 | | | | 450 | | |
| 51.7 | | | | | 56 | |
| 52.2 | | | | 275 | | |
| 54.4 | | | | 78 | 23 | 10 |

The above description of illustrated embodiments, including what is described in the Abstract, is not intended to be exhaustive or to limit the embodiments to the precise forms disclosed. Although specific embodiments and examples are described above for illustrative purposes, various equivalent modifications can be made without departing from the spirit and scope of the disclosure, as will be recognized by those skilled in the relevant art. The teachings provided above of the various embodiments can be applied to other systems, methods and/or processes to dispense high viscosity compositions into porous substrates and devices produced thereby, not only the exemplary systems, methods and devices generally described above.

For instance, the detailed description above has set forth various embodiments of the systems, processes, methods and/or devices via the use of block diagrams, schematics, flow charts and examples. Insofar as such block diagrams, schematics, flow charts and examples contain one or more functions and/or operations, it will be understood by those skilled in the art that each function and/or operation within such block diagrams, schematics, flowcharts or examples can be implemented, individually and/or collectively, by a wide range of system components, hardware, software, firmware, or virtually any combination thereof.

In certain embodiments, the systems used or devices produced may include fewer structures or components than in the particular embodiments described above. In other embodiments, the systems used or devices produced may include structures or components in addition to those described herein. In further embodiments, the systems used or devices produced may include structures or components that are arranged differently from those described herein. For example, in some embodiments, there may be additional heaters and/or mixers in the dispensing system to provide effective control the temperature of the composition. Further, in implementation of procedures or methods described herein, there may be fewer operations, additional operations, or the operations may be performed in different order from those described herein. For example, in a continuous web-based manufacturing system, all layers of the final structure, including the porous substrate, may be associated before the composition is dispensed into the porous substrate, i.e., fill may take place late in the process during which a transdermal device is made. Removing, adding, or rearranging system or device components, or operational aspects of the processes or methods, would be well within the skill of one of ordinary skill in the relevant art in light of this disclosure.

Regarding control and operation of the systems and processes, or design of the transdermal delivery devices, in certain embodiments, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof. Accordingly, designing the circuitry and/or writing the code for the software and or firmware would be well within the skill of one of ordinary skill in the art in light of this disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for dispensing into a porous substrate a composition comprising a highly viscous liquid, a sol or a sol-forming material including at least one cellulose derivative via a conduit having an inlet, an outlet, a first portion spaced between the inlet and the outlet, and a second portion spaced between the first portion and the outlet, the method comprising:
providing at the outlet end of the conduit the porous substrate;
adjusting a temperature of the first portion of the conduit to a first temperature of at least about 35° C., said temperature sufficient to transform a viscosity of the composition comprising the highly viscous liquid, the sol or the sol-forming material including at least one cellulose derivative from a high viscosity to a low viscosity;
moving the composition comprising the highly viscous liquid, the sol or the sol-forming material including at least one cellulose derivative from the inlet end of the conduit to the outlet end of the conduit; and
dispensing the composition from the outlet end of the conduit into the porous substrate.

2. The method of claim 1 wherein adjusting a temperature of the first portion of the conduit to a first temperature of at least about 35° C. includes adjusting the temperature of the first portion of the conduit sufficiently to transform a viscosity of the composition from a high viscosity of between about 2,500 centipoise and about 10,000 centipoise to a low viscosity.

3. The method of claim 1 wherein adjusting a temperature of the first portion of the conduit to a first temperature of at least about 35° C. includes adjusting the temperature of the first portion of the conduit sufficiently to transform a viscosity of the composition from a high viscosity to a low viscosity of between about 0 centipoise and 500 centipoise at some point in the first portion.

4. The method of claim 1 wherein adjusting a temperature of the first portion of the conduit to a first temperature of at least about 35° C. includes adjusting the temperature of the first portion of the conduit sufficiently to transform a viscosity of the composition from a high viscosity to a low viscosity of between about 50 centipoise and about 150 centipoise at some point in the first portion.

5. The method of claim 1 wherein adjusting a temperature of the first portion of the conduit to a first temperature of at least about 35° C. includes adjusting the temperature of the first portion of the conduit to between about 45° C. and about 70° C.

6. The method of claim 1 wherein adjusting a temperature of the first portion of the conduit to a first temperature of at least about 35° C. includes adjusting the temperature of the first portion of the conduit to between about 40° C. and about 60° C.

7. The method of claim 1 wherein adjusting a temperature of the first portion of the conduit to a first temperature of at least about 35° C. includes adjusting the temperature of the first portion of the conduit to between about 40° C. and about 50° C.

8. The method of claim 1 wherein adjusting a temperature of the first portion of the conduit to a first temperature of at least about 35° C. includes adjusting the temperature of the first portion of the conduit to between about 50° C. and about 60° C.

9. The method of claim 1 wherein adjusting a temperature of the first portion of the conduit to a first temperature of at least about 35° C. includes adjusting the temperature of the first portion of the conduit to between about 52° C. and about 55° C.

10. The method of claim 1 wherein adjusting a temperature of the first portion of the conduit to a first temperature of at least about 35° C. includes adjusting the temperature of the first portion of the conduit to between about 40° C. and about 43° C.

11. The method of claim 2, further comprising:
adjusting a temperature of the second portion of the conduit to a second temperature.

12. The method of claim 11 wherein adjusting a temperature of the second portion of the conduit to a second temperature includes adjusting the temperature of the second portion sufficiently to maintain the viscosity of the composition at the low viscosity at the outlet end of the conduit.

13. The method of claim 11 wherein adjusting a temperature of the second portion of the conduit to a second temperature includes adjusting the temperature of the second portion sufficiently such that the viscosity of the composition at the outlet end of the conduit is between about 50 centipoise and about 150 centipoise.

14. The method of claim 11 wherein adjusting a temperature of the second portion of the conduit to a second temperature includes adjusting the temperature of the second portion to be less than or equal to the first temperature.

15. The method of claim 11 wherein adjusting a temperature of the second portion of the conduit to a second temperature includes adjusting the temperature of the second portion to be greater than or equal to the first temperature.

16. The method of claim 11 wherein adjusting a temperature of the second portion of the conduit to a second temperature includes adjusting the temperature of the second portion of the conduit to be greater than 35° C.

17. The method of claim 11 wherein adjusting a temperature of the second portion of the conduit to a second temperature includes adjusting the temperature of the second portion of the conduit to between about 35° C. and about 70° C.

18. The method of claim 11 wherein adjusting a temperature of the second portion of the conduit to a second temperature includes adjusting the temperature of the second portion of the conduit to between about 40° C. and about 50° C.

19. The method of claim 11 wherein adjusting a temperature of the second portion of the conduit to a second temperature includes adjusting the temperature of the second portion conduit to between about 50° C. and about 60° C.

20. The method of claim 11 wherein adjusting a temperature of the second portion of the conduit to a second temperature includes adjusting the temperature of the second portion of the conduit to between about 49° C. and about 52° C.

21. The method of claim 11 wherein adjusting a temperature of the first portion of the conduit to a first temperature includes adjusting the temperature of the first portion of the conduit to between about 52° C. and about 55° C. and adjusting a temperature of the second portion of the conduit to a second temperature includes adjusting the temperature of the second portion of the conduit to between about 49° C. and about 52° C.

22. The method of claim 11 wherein adjusting a temperature of the first portion of the conduit to a first temperature includes adjusting the temperature of the first portion of the conduit to between about 49° C. and about 52° C. and adjusting a temperature of the second portion of the conduit to a second temperature includes adjusting the temperature of the second portion of the conduit to between about 52° C. and about 55° C.

23. The method of claim 11 wherein adjusting a temperature of the first portion of the conduit to a first temperature and adjusting a temperature of the second portion of the conduit to a second temperature includes adjusting the temperature of the first portion of the conduit and the temperature of the second portion of the conduit to between about 49° C. and about 53° C.

24. The method of claim 11 wherein adjusting a temperature of the first portion of the conduit to a first temperature and adjusting a temperature of the second portion of the conduit to a second temperature includes adjusting the temperature of the first portion of the conduit and the temperature of the second portion of the conduit to between about 39° C. and about 43° C.

25. The method of claim 1, further comprising:
providing the composition including the cellulose derivative in the form of at least one of an alkylcellulose ether or a modified alkylcellulose ether to the inlet of the conduit.

26. The method of claim 1, further comprising:
providing the composition including the cellulose derivative in the form of at least one of a hydroxypropyl cellulose, a hydroxyethyl cellulose, a hydroxypropylmethyl cellulose, or a carboxymethyl cellulose to the inlet of the conduit.

27. The method of claim 1, further comprising:
providing the composition including the cellulose derivative in the form of a hydroxypropyl cellulose to the inlet of the conduit.

28. The method of claim 1, further comprising:
providing the composition including the cellulose derivative in the form of a hydroxypropyl cellulose in a percent concentration (w/w×100) between about 1% and about 2.5% to the inlet of the conduit.

29. The method of claim 1, further comprising:
providing the composition including the cellulose derivative in the form of a hydroxypropyl cellulose in a percent concentration (w/w×100) between about 1.5% and about 2% to the inlet of the conduit.

30. The method of claim 1, further comprising:
providing the composition including the cellulose derivative in the form of a mixture of hydroxypropyl cellulose and a second cellulose derivative to the inlet of the conduit.

31. The method of claim 1, further comprising:
providing the composition including the cellulose derivative in the form of a mixture of hydroxypropyl cellulose and hydroxyethyl cellulose to the inlet of the conduit.

32. The method of claim 1, further comprising:
providing the composition including the cellulose derivative in the form of mixture of hydroxypropyl cellulose and hydroxyethyl cellulose to the inlet of the conduit, wherein a ratio of percent concentration (w/w×100) of hydroxypropyl cellulose to percent concentration (w/w×100) of hydroxyethyl cellulose is between about 4:1 and about 2:1.

33. The method of claim 1, further comprising:
providing the composition including the cellulose derivative in the form of mixture of hydroxypropyl cellulose and hydroxyethyl cellulose to the inlet of the conduit, wherein the ratio of percent concentration (w/w×100) of hydroxypropyl cellulose to percent concentration (w/w×100) of hydroxyethyl cellulose is between about 3.5:1 and about 2.5:1.

34. The method of claim 1, further comprising:
providing the composition including the cellulose derivative in the form of mixture of hydroxypropyl cellulose and hydroxyethyl cellulose to the inlet of the conduit, wherein the ratio of percent concentration (w/w×100) of hydroxypropyl cellulose to percent concentration (w/w×100) of hydroxyethyl cellulose is about 3:1.

35. The method of claim 1, further comprising:
providing the composition including the cellulose derivative in the form of mixture of hydroxypropyl cellulose and hydroxyethyl cellulose to the inlet of the conduit, wherein the percent concentration (w/w×100) of hydroxypropyl cellulose is about 1.5% and the percent concentration (w/w×100) of hydroxyethyl cellulose is about 0.5%.

36. The method of claim 1, further comprising:
providing the composition in the form of an electrolyte composition to the inlet of the conduit.

37. The method of claim 1, further comprising:
providing the composition which further comprises a biologically active agent to the inlet of the conduit.

38. The method of claim 1, further comprising:
providing the composition which further comprises a biologically active agent selected from the group consisting of—caine-type active agents to the inlet of the conduit.

39. The method of claim 1, further comprising:
providing the composition which further comprises a biologically active agent in the form of lidocaine to the inlet of the conduit.

40. The method of claim 1, further comprising:
providing the composition which further comprises a biologically active agent in the form of a combination of lidocaine and epinephrine to the inlet of the conduit.

41. The method of claim 1 wherein dispensing the composition from the outlet end of the conduit includes dispensing the composition having a viscosity that is between about 50 centipoise and about 150 centipoise.

42. The method of claim 1, further comprising:
providing the composition to the inlet of the conduit from a pressurized dispensing reservoir; and
regulating a flow of the composition from the outlet end of the conduit via a valve positioned at least proximate the second portion of the conduit.

43. The method of claim 1, further comprising:
providing the composition to the inlet of the conduit from a metering pump; and
regulating a flow of the composition from the outlet end of the conduit by adjusting the metering pump, thereby regulating a flow of the composition to the inlet of and through the conduit.

44. The method of claim 1, further comprising:
dispensing the composition into a holding portion of a device for iontophoretic delivery of an active agent to a biological interface; and
allowing the composition to return to an ambient temperature.

45. The method of claim 44 wherein dispensing the composition into a holding portion of a device for iontophoretic delivery includes dispensing the composition into a matrix.

46. The method of claim 1 wherein the porous substrate is a component of a reservoir of a device for delivery of an active agent to or through a biological interface.

47. The method of claim 1 wherein the porous substrate is a component of a reservoir of a device for transdermal delivery of an active agent to or through a biological interface.

48. The method of claim 1 wherein the porous substrate is a component of a reservoir of a device for iontophoretic delivery of an active agent to or through a biological interface.

49. A system for dispensing a highly viscous liquid, a sol or a sol-forming composition comprising at least one cellulose derivative, the system comprising:
a conduit for transporting the composition, the conduit comprising an inlet, an outlet, a first portion positioned between the inlet and the outlet and a second portion positioned between the first portion and the outlet;
a first heater positioned to heat the composition in at least part of the first portion of the conduit to a first temperature; and
a valve mechanism operable to control a rate of dispensing the composition from the conduit.

50. The system of claim 49 further comprising:
a second heater positioned to heat the composition in at least part of the second portion of the conduit to a second temperature.

51. The system of claim 49 wherein the first heater includes at least one of a heat exchanger or a heater element.

52. The system of claim 50 wherein the second temperature is less than or equal to the first temperature.

53. The system of claim 50 wherein the second temperature is greater than or equal to the first temperature.

54. The system of claim 50 wherein the second heater includes at least one of a heat exchanger or a heater element.

55. The system of claim 50 wherein at least a portion of the second heater is embedded in at least a portion of the valve mechanism.

56. The system of claim 49, further comprising:
a mixer at least proximate the first portion.

57. The system of claim 56 wherein the mixer is a static mixer.

58. The system of claim 56 wherein the mixer is a dynamic mixer.

59. The system of claim 49, further comprising:
a dispensing reservoir to store the composition to be dispensed, the dispensing reservoir fluidly communicably coupled to the inlet of the conduit via a fluid-tight connection.

60. The system of claim 59 wherein the dispensing reservoir is a pressurized reservoir.

61. The system of claim 59 wherein the dispensing reservoir stores the composition that includes the cellulose derivative in the form of a hydroxypropyl cellulose.

62. The system of claim 59 wherein the dispensing reservoir stores the composition that includes the cellulose derivative in the form of a mixture of hydroxypropyl cellulose and hydroxyethyl cellulose.

63. The system of claim 59 wherein the dispensing reservoir stores the composition that further comprises at least one biologically active agent.

64. The system of claim 59 wherein the dispensing reservoir stores the composition that further comprises at least one biologically active agent selected from the—caine-type active agents.

65. A system for dispensing a highly viscous liquid, a sol or a sol-forming composition comprising at least one cellulose derivative, the system comprising:
a conduit for transporting the composition, the conduit comprising an inlet, an outlet, a first portion positioned between the inlet and the outlet and a second portion positioned between the first portion and the outlet;
a metering pump to supply the composition to the inlet of the conduit; and
a first heater positioned to heat the composition in at least part of the first portion of the conduit to a first temperature.

66. The system of claim 65 further comprising:
a second heater positioned to heat the composition in at least part of the second portion of the conduit to a second temperature.

67. A composition, comprising:
a first cellulose derivative;
a second cellulose derivative; and
a biologically active agent;
wherein the first cellulose derivative is hydroxypropyl cellulose; and
wherein the second cellulose derivative differs from the first cellulose derivative.

68. The composition of claim 67 wherein the second cellulose derivative is selected from hydroxyethyl cellulose, hydroxypropylmethyl cellulose, or carboxymethyl cellulose.

69. The composition of claim 68 wherein the second cellulose derivative is hydroxyethyl cellulose.

70. The composition of claim 67 wherein the biologically active agent is selected from the group consisting of—caine-type active agents.

71. The composition of claim 67 wherein the biologically active agent is lidocaine.

72. The composition of claim 67 wherein the biologically active agent is a mixture of lidocaine and epinephrine.
